(19) United States
(12) Patent Application Publication
(10) Pub. No.: US 2025/0161645 A1
(43) Pub. Date: May 22, 2025

(54) OXYGEN DELIVERY TO A WOUND

(71) Applicant: T.J.Smith and Nephew,Limited, Hull (GB)

(72) Inventors: Anthony Colin Dagger, York (GB); Victoria Jody Hammond, Hull (GB)

(21) Appl. No.: 18/842,980

(22) PCT Filed: Feb. 28, 2023
(86) PCT No.: PCT/EP2023/054966

§ 371 Date: Aug. 30, 2024

(30) Foreign Application Priority Data
Mar. 1, 2022 (GB) 2202788.2

(51) Int. Cl. A61M 35/00; A61F 13/01; A61F 13/05; A61L 15/20; A61L 15/28; A61L 15/60

(52) U.S. Cl. CPC A61M 35/30; A61F 13/01012; A61F 13/01029; A61F 13/01042; A61F 13/05; A61L 15/20; A61L 15/28; A61L 15/60; A61M 35/10; A61M 2202/0208

(57) ABSTRACT

Disclosed embodiments relate to a wound dressing which can contain and release oxygen. The wound dressing may include one or more perfluorocarbon layers, a membrane electrolytic assembly, one or more dressing layers, and a durable binding layer. The one or more perfluorocarbon layers may contain nonafluorohexyltriethoxysilane, 1H, 1H, 2H, 2H-perfluorodecyltriethoxysilane, 1H, 1H, 2H, 2H-perfluorooctyltriethoxysilane, or pentafluorophenyltriethoxysilane. The wound dressing may also include other layers.

### BACKGROUND

### Technical Field

Disclosed herein are materials, devices, methods, and systems, such as therapeutic compositions, wound care materials, their uses, and methods of treatment therewith. In some examples, the materials, devices, and systems described herein comprise a wound dressing configured for oxygen delivery and/or the delivery of other actives.

### Description of the Related Art

Oxygen is a well-known molecule with multiple biological functions. The delivery of oxygen to the periwound area and/or the wound edge may target, for example cell proliferation, angiogenesis, and protein synthesis. Oxygen demonstrates a potent effect on tissue and increased amounts of oxygen may support the acceleration of healing in wounds, particularly chronic wounds. Increased wound tissue oxygenation can initiate regeneration at the wound site as well as positively impact other treatments.

Diminished supply of oxygen may lead to vascular damage, such as endothelial dysfunction and vascular inflammation. Vascular damage may also lead to decreased blood flow to the extremities, thereby potentially causing the diabetic patient to be more likely to develop neuropathy and non-healing ulcers, and to be at a greater risk for lower limb amputation.

Consequently, there is a need for improved mechanisms of delivering an effective dose of oxygen to a wound. Under normal conditions, atmospheric concentrations of oxygen are insufficient to diffuse and fully saturate a wound. Accordingly, it may be difficult to maintain high concentrations of oxygen within a wound dressing or other similar structure for a prolonged period of time. Therefore, a device or a wound dressing having one or more layers containing more stable compositions may effectively capture large quantities of oxygen and release oxygen upon contact with a wound, for the stable and sustained delivery of oxygen to biological tissues. Of particular interest are mechanisms of delivering oxygen in combination with use of a wound dressing, particularly a negative pressure wound dressing and/or while undergoing negative pressure wound therapy and/or other appropriate therapies.

### SUMMARY

Embodiments of the present disclosure relate to materials, devices, methods, and systems for wound treatment. Some disclosed embodiments relate to materials, devices, methods, and systems for delivering oxygen to a wound. It will be understood by one of skill in the art that application of the materials, devices, methods, and systems described herein are not limited to a particular tissue, particular location on the body, or a particular injury.

In some configurations, a wound dressing for treating a wound includes one or more perfluorocarbon layers, a membrane electrolytic assembly, one or more dressing layers, and a cover layer.

In certain examples, a wound dressing assembly for treating a wound may comprise a perfluorocarbon layer configured to deliver oxygen to a wound, the perfluorocarbon layer comprising perfluorocarbon bound to a cellulose fiber. The wound dressing assembly may comprise a cover layer configured to form a seal around the wound. The wound dressing assembly may comprise a membrane electrolytic assembly configured to deliver oxygen to the perfluorocarbon layer. In certain examples, the wound dressing assembly may comprise a wound contact layer and/or an absorbent layer and/or a transmission layer and/or an obscuring layer. In some examples, the perfluorocarbon layer may comprise nonafluorohexyltriethoxysilane. In some examples, the perfluorocarbon layer may comprise pentafluorophenyltriethoxysilane. The perfluorocarbon layer may contain 1H, 1H, 2H, 2H-perfluorodecyltriethoxysilane and/or 1H, 1H, 2H, 2H-perfluorooctyltriethoxysilane. In certain examples, the wound dressing assembly may comprise a gel and/or a hydrogel. In some examples, the perfluorocarbon layer may be attached to the membrane electrolytic assembly.

In some examples, a method for treating a wound may comprise applying a wound dressing to a wound, the wound dressing comprising a perfluorocarbon layer, transporting oxygen to the perfluorocarbon layer such that the perfluorocarbon layer stores the oxygen; and delivering oxygen to the wound from the perfluorocarbon layer. In certain examples, the oxygen may be provided by a membrane electrolytic assembly.

In certain examples, a kit for treating a wound may comprise a perfluorocarbon layer configured to delivery oxygen to a wound; a wound dressing configured to distribute oxygen; and a membrane electrolytic assembly. The kit may further include a sealing strip.

The wound dressing can include one or more of the following features. The wound dressing can further include a cover layer configured to form a seal around the wound. The one or more dressing layers can include a plurality of fibers, and 80% to 90% of the plurality of fibers extend horizontally, or substantially horizontally. The cover layer can be moisture vapor permeable. The cover layer can have an outer perimeter larger than an outer perimeter of the activator layer, thereby defining a border region between the outer perimeter of the cover layer and the outer perimeter of the activator layer. At least a portion of the border region of the cover layer can be configured to be sealed to the skin around the wound. The wound dressing can further include an obscuring layer. The one or more perfluorocarbon layers can contain nonafluorohexyltriethoxysilane. The one or more perfluorocarbon layers can contain pentafluorophenyltriethoxysilane. The one or more perfluorocarbon layers can contain 1H, 1H, 2H, 2H-perfluorodecyltriethoxysilane. The one or more perfluorocarbon layers can contain 1H, 1H, 2H, 2H-perfluorooctyltriethoxysilane.

In some configurations, a method for treating a wound includes applying a wound dressing to the wound. The wound dressing includes one or more perfluorocarbon layers configured to horizontally and/or vertically wick fluid.

The method of the preceding paragraph can include one or more of the following features. The method can further include capturing and releasing oxygen within the one or more perfluorocarbon layers. The wound dressing can further include a cover layer, wherein the cover layer has an outer perimeter larger than an outer perimeter of the acid providing layer, thereby defining a border region between the outer perimeter of the cover layer and the outer perimeter of the one or more perfluorocarbon layers, and sealing at least a portion of the border region of the cover layer to the skin around the wound. The wound dressing can further include an obscuring layer positioned between the one or more perfluorocarbon layers and the cover layer.

In some configurations, a wound dressing for treating a wound includes a cover layer, one or more perfluorocarbon layers, one or more dressing layers, and a cover layer. The cover layer is configured to form a seal around the wound.

The wound dressing of the preceding paragraph can include one or more of the following features. The cover layer can be moisture vapor permeable. The cover layer can have an outer perimeter larger than an outer perimeter of the one or more perfluorocarbon layers, thereby defining a border region between the outer perimeter of the cover layer and the outer perimeter of the one or more perfluorocarbon layers. At least a portion of the border region of the cover layer can be configured to be sealed to the skin around the wound. The wound dressing can include an obscuring layer positioned between the one or more perfluorocarbon layers and the cover layer. The wound dressing can contain an absorption layer. The wound dressing can contain a transmission layer. The wound dressing can contain a masking or obscuring layer.

In some configurations, a wound dressing includes one or more perfluorocarbon layers comprising a plurality of through-holes through the thickness of the one or more perfluorocarbon layers.

The wound dressing of the preceding paragraph can include one or more of the following features. The wound dressing can further include a cover layer configured to form a seal around the wound. The cover layer can be moisture vapor permeable. The one or more perfluorocarbon layers can include a mesh. The wound dressing can further include an obscuring layer.

Alternative or additional embodiments described herein provide a composition comprising one or more of the features of the foregoing description or of any description elsewhere herein. Alternative or additional embodiments described herein provide a wound contact layer comprising one or more of the features of the foregoing description or of any description elsewhere herein. Alternative or additional embodiments described herein provide a wound dressing comprising one or more of the features of the foregoing description or of any description elsewhere herein.

Alternative or additional embodiments described herein provide a wound treatment system comprising one or more of the features of the foregoing description or of any description elsewhere herein.

Alternative or additional embodiments described herein provide a method of treating a wound comprising one or more of the features of the foregoing description or of any description elsewhere herein.

### BRIEF DESCRIPTION OF THE DRAWINGS

FIG. 1A is an illustration of a negative pressure wound dressing system;

FIG. 1B illustrates an embodiment of a negative pressure wound treatment system employing a pump, a flexible fluidic connector and a wound dressing capable of absorbing and storing wound exudate;

FIG. 1C illustrates an embodiment of a negative pressure wound treatment system employing a flexible fluidic connector and a wound dressing capable of absorbing and storing wound exudate;

FIG. 1D illustrates a cross section of an embodiment of a fluidic connector connected to a wound dressing;

FIG. 2 is an illustration of an example of a perfluorocarbon dressing wound therapy system;

FIG. 3 illustrates an embodiment of a perfluorocarbon dressing wound therapy system with a wound contact layer;

FIG. 4 illustrates an embodiment of a perfluorocarbon dressing wound treatment system with an absorbent layer;

FIG. 5 illustrates an embodiment of a perfluorocarbon dressing wound treatment system with an absorbent layer and a masking layer;

FIG. 6 illustrates an embodiment of a perfluorocarbon dressing wound treatment system with a transmission layer and a close-up view of the wound contacting layer;

FIG. 7 illustrates an example of a silanisation reaction binding a perfluorocarbon layer to a dressing layer;

FIG. 8A illustrates a line graph of the relative percent oxygen absorption for multiple different perfluorocarbon samples bound to DURAFIBER;

FIG. 8B illustrates a bar graph of the relative percent oxygen absorption for multiple different perfluorocarbon samples bound to DURAFIBER;

FIG. 8C illustrates a line graph of the relative percent oxygen absorption for multiple different perfluorocarbon samples bound to DURAFIBER;

FIG. 8D illustrates a bar graph of the relative percent oxygen absorption for multiple different perfluorocarbon samples bound to DURAFIBER;

FIG. 8E illustrates a line graph of the relative percent oxygen absorption for multiple different perfluorocarbon samples bound to DURAFIBER;

FIG. 9A illustrates a line graph of the relative percent oxygen release for multiple different concentrations of nonafluorohexyltriethoxysilane bound to DURAFIBER;

FIG. 9B illustrates a bar graph of the relative percent oxygen release for multiple different concentrations of nonafluorohexyltriethoxysilane bound to DURAFIBER;

FIG. 10A illustrates a study focusing on the effect of oxygen-loaded nonafluorohexyltriethoxysilane dressings on wound closure over time;

FIG. 10B illustrates a study focusing on the effect of oxygen-loaded nonafluorohexyltriethoxysilane dressings on wound closure over time;

FIG. 11A displays tissue samples from an ex vivo study on perfluorocarbon wound dressings to determine relative oxygen delivery and wound closure rate over time;

FIG. 11B displays a bar graph from an ex vivo study on perfluorocarbon wound dressings to determine relative oxygen delivery and wound closure rate over time; and

FIG. 12 illustrates an ex vivo study on perfluorocarbon wound dressings focusing on nonafluorohexyltriethoxysilane.

### DETAILED DESCRIPTION

### Overview

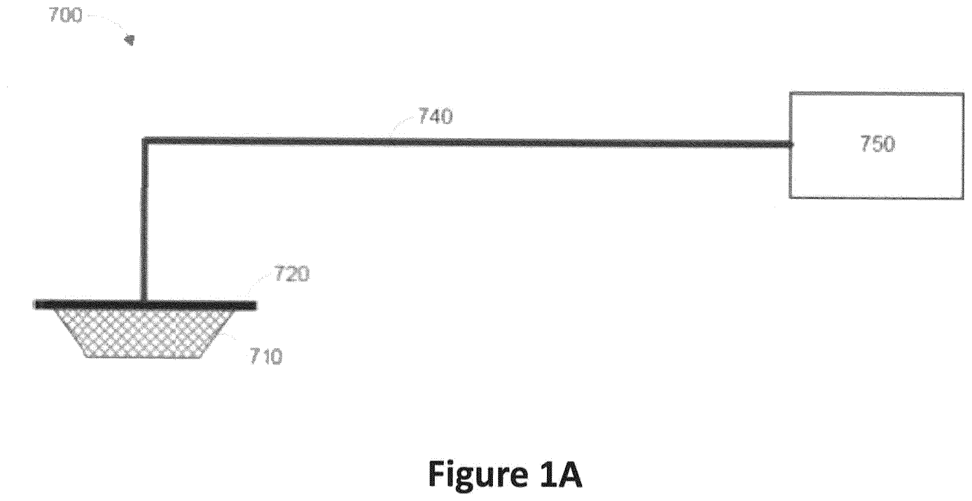

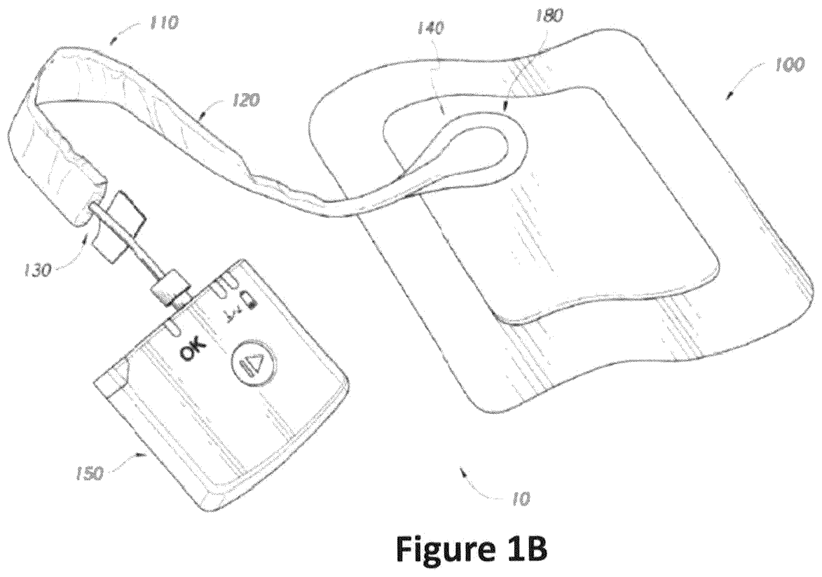

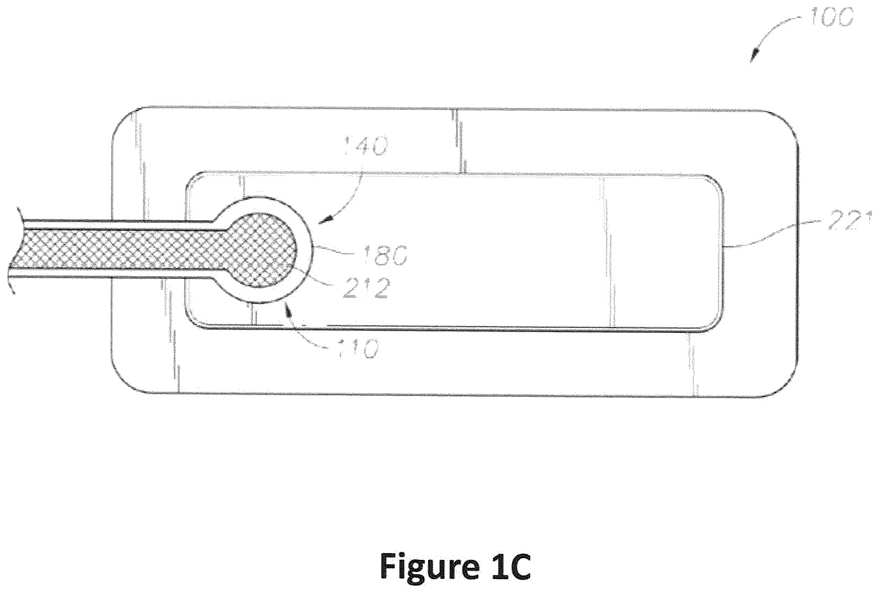

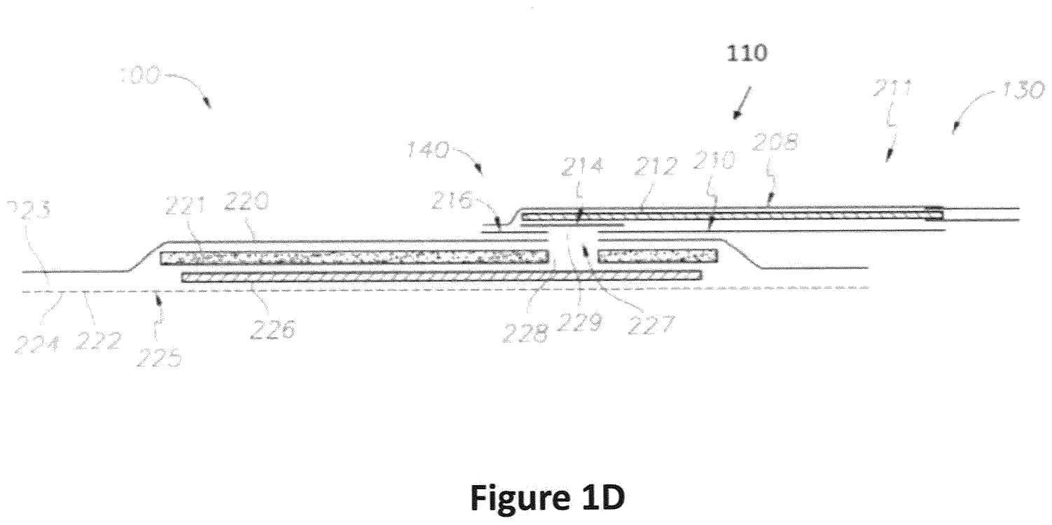

Embodiments described herein relate to materials, apparatuses, methods, and systems that incorporate, or comprise, or utilize one or more compositions and/or materials that effectively store and release gases (e.g. oxygen) over time upon activation. Embodiments herein may be directed toward a device and/or a wound dressing having one or more layers containing compositions and/or materials that effectively store and release oxygen over time upon activation, for example a perfluorocarbon layer. Such perfluorocarbon layers will be described in greater detail elsewhere in the specification, such as below. The one or more perfluorocarbon layers may be utilized as a stand-alone component for separately positioning at a wound site, or may be incorporated into any number of multi-layer wound dressings and wound treatment apparatuses, such as described herein below with respect to FIGS. 1 through 11. Embodiments of the present disclosure are generally applicable to use under ambient conditions, in negative pressure or reduced pressure therapy systems, or in compression therapy systems. Some of the preferred embodiments described herein incorporate, or comprise, or utilize one or more perfluorocarbon layers. As will be described in greater detail below, such a perfluorocarbon layer may be formed by functionalizing cellulose fibers (such as DURAFIBER, sold by Smith+Nephew) with various perfluorocarbons. As will be understood by one of skill in the art, DURAFIBER is an absorbent cellulose gelling fiber which combines natural strengthening cellulose fibers with gelling cellulose ethyl sulphonate fibers to form a finely spun matrix that forms a soft cohesive gel upon contact with liquid, such as wound exudate. As will be explained further below, such perfluorocarbons can be coupled to cellulose via silane coupling. Considering silane on glass fibers, silane OH groups form by hydrolysis in aqueous alcohol loading media condense with the OH moieties present on the glass surface: ≡Si—OH+OH—R↔R—O—Si≡+H₂O. Plueddeman, E.P. Silane Coupling Agents, 2^{nd} ed.; Plenum Press, NY 1991. Similarly, hydrolyzed silane adsorbed onto the cellulose fibers as described herein may be heated in order to remove the aqueous alcohol loading solvent, thereby leading to binding via silane coupling.

As will be understood by one of skill in the art, perfluorocarbons may be loaded with oxygen and act as oxygen carriers. Such one or more perfluorocarbon layers, once loaded with oxygen, may possess one or more of the following functional features: inflammation-related activities, blood flow-related activities, antimicrobial, anti-planktonic and anti-biofilm activities, ease of application or/and removal as one piece, cuttability/tearability, conformability to the three-dimensional contour of a wound surface, durability to wear, compatibility with negative pressure wound therapy or/and compression wound therapy, exudate management, capability of facilitating autolytic debridement of wounds, capability of promoting wound healing, and self-indication of compositional or functional changes. The antimicrobial activities, such as in vitro antimicrobial activities, can include one or more of the following: broad-spectrum antimicrobial activity, anti-biofilm activity, rapid speed of kill against microorganisms, sustained kill against microorganisms; and the microorganisms can include one or more of the following: Gram-negative bacteria, Gram-positive bacteria, fungi, yeasts, viruses, algae, archaea and protozoa.

Certain preferred embodiments described herein provide a wound treatment system. Such a wound treatment system may comprise perfluorocarbon layers, configured to be sized for positioning over a wound and/or the periwound area. One of skill in the art will understand that when an apparatus/dressing/layer is described as being placed on or over a wound, such an apparatus/dressing/layer may extend over and treat the periwound area. In some instances, stimulation of the periwound area and/or the wound edge may play a role in initiating the wound healing process, and the wound healing process can be activated through the delivery of oxygen to the periwound area and/or the wound edge. The delivery of oxygen to the periwound area and/or the wound edge may target, for example cell proliferation, angiogenesis, and protein synthesis. The wound treatment systems described herein may further comprise a secondary wound dressing configured to be separately positioned over the perfluorocarbon layers. The perfluorocarbon layers may have an adhesive adhered to the lower surface; and the adhesive can be configured such that the perfluorocarbon layers may be placed in proximity to the wound. The secondary wound dressing, if used, may adhere to skin surrounding the wound and may have the same size or may be larger than the perfluorocarbon layers, such that the perfluorocarbon layers will touch or be placed in proximity to the wound and/or the periwound area. The secondary wound dressing can be alternatively or additionally configured to form a seal to skin surrounding the wound so that the perfluorocarbon layers will touch or be placed in proximity to the wound. The wound treatment system may further comprise a source of negative pressure configured to supply negative pressure through the secondary wound dressing and through the wound contact layer to the wound.

Certain embodiments described herein may provide a multi-layered wound dressing, such as described herein the specification with respect to FIGS. 1 through 12. Such a multi-layered wound dressing may incorporate the one or more perfluorocarbon layers as component layers thereof or, alternatively, may comprise a composite or laminate including the one or more perfluorocarbon layers as part of one of the component layers thereof. The process of constructing a perfluorocarbon layer will be described further elsewhere in the specification. The multi-layered wound dressing may comprise: perfluorocarbon layers as described above or described elsewhere herein; a transmission layer and/or absorbent layer over/under the one or more perfluorocarbon layers; a membrane electrolytic assembly over the one or more perfluorocarbon layers; a wound contact layer under the one or more perfluorocarbon layers; an absorption layer; one or more dressing layers; a masking or obscuring layer; and a cover layer over the transmission layer and/or absorbent layer. The wound dressing may further comprise a negative pressure port positioned on or above the cover layer. The one or more perfluorocarbon layers may have a perimeter shape that is substantially the same as a perimeter shape of the cover layer. Alternatively, the one or more perfluorocarbon layers may have a perimeter shape that is smaller than a perimeter shape of the cover layer.

One of skill in the art will understand that perfluorocarbon functionalized hydrogel, gel, and/or xerogel compositions, such as any disclosed herein this “Overview” section or elsewhere in the specification, may be present within the one or more perfluorocarbon layers in any suitable form, such as via adsorption, absorption, chemical and/or physical attachment entanglement, and/or via powder form. One of skill in the art will further understand that reactive compositions, such as any disclosed herein this section or elsewhere in the specification may be incorporated into any suitable absorbent layer disclosed herein this section or elsewhere in the specification by any suitable means, and/or any suitable transmission layer disclosed herein this section or elsewhere in the specification, and/or any foam layer disclosed herein this section or elsewhere in the specification.

In certain embodiments, the wound treatment systems and multi-layered wound dressings disclosed above or disclosed elsewhere herein the specification may incorporate or comprise perfluorocarbon layers. As described herein this section or elsewhere in the specification, particularly below, the perfluorocarbon layers may be configured to be activated to release oxygen. At least a portion of the released oxygen may be released, for example by diffusion. To facilitate release and diffusion of oxygen, the perfluorocarbon layers may be placed proximate to the wound.

Some preferred embodiments described herein the specification provide a method to treat a wound, intact tissue, or other suitable location. Such a method may include placing perfluorocarbon layers, either separately or by placing a multi-layered wound dressing having perfluorocarbon layers, over the wound. The method may comprise adhering the separate perfluorocarbon layers and/or the multi-layer wound dressing having one or more perfluorocarbon layers to healthy skin around the wound. Such a method may further comprise one or more of the following steps: A further wound dressing can be placed over the separate perfluorocarbon layers or multi-layered wound dressing having the nitric oxide generating layers that is placed over the wound. Wound exudate, or any moist or aqueous medium other than wound exudate, may be provided to reach and/or touch the perfluorocarbon layers. Wound exudate, or any moist or aqueous medium other than wound exudate may be diffused or wicked into the wound dressing incorporating the perfluorocarbon layers or into a wound dressing provided over the perfluorocarbon layers. Negative pressure may be applied to the separate perfluorocarbon layers or multi-layered wound dressing having the nitric oxide generating layers, such that wound exudate is suctioned into the perfluorocarbon layers directly, or into the wound dressing incorporating the perfluorocarbon layers, or into a wound dressing provided over the perfluorocarbon layers.

One of skill in the art will understand that wound dressings, devices and systems disclosed herein this “Overview” section or elsewhere in the specification may include one or more layers, compositions, materials or components that release gases other than oxygen in addition to the perfluorocarbon layers, compositions or materials. For example, a wound dressing or a device can include one or more layers that effectively generate vasodilatory agents, such as carbon monoxide or hydrogen sulfide, over time upon activation.

One of skill in the art will further understand that carbon monoxide and/or hydrogen sulfide may be used in combination with an oxygen delivery element (such as a layer) where suitable. Further details regarding generation and delivery of carbon monoxide and/or hydrogen sulfide may be found in chapter six of the text Inorganic and Organometallic Transition Metal Complexes with Biological Molecules and Living Cells, ISBN 978-0-12-803814-7, which is hereby incorporated by reference. For example, hydrogen sulfide may be generated from elements/layers that contain cleavable/releasable hydrogen sulfide, diallyl thiosulfinate, GYY4137, S-Mesalamine ATB-429, S-Naproxen ATB-346, S-Diclofenac ATB-337/ACS-15. For example, carbon monoxide may be generated from elements/layers that provide of complexes of carbon monoxide bound to suitable metals such as chromium, molybdenum, tungsten, manganese, rhenium, iron, ruthenium, cobalt, rhodium, and iridium. Such complexes may be enzymatically triggered to release carbon monoxide, photo-cleavable, and/or responsive to interaction with a suitable ligand to induce release of carbon monoxide.

Perfluorocarbons are chemically inert volatile liquids which possess a high respiratory gas-dissolving capability. Perfluorocarbons have been employed as both oxygen storage and oxygen delivery vehicles to selectively transport oxygen to a wound. In some cases, due to their ability to store and release oxygen, perfluorocarbons have been breathed in directly. Perfluorocarbons have been investigated for their ability to deliver oxygen to ischemic secondary burns on porcine models with promising results, showing accelerated epithelialization and collagen deposition of the wound and the promotion of vasculogenesis. Cell culture experiments have shown that MAFC oxygenating gels improved cellular functions involved with wound healing such as total DNA synthesis, cell metabolism, and cell migration under hypoxic conditions in both fibroblasts and keratinocytes. In rat wound studies, perfluorocarbons improved re-epithelialization and collagen synthesis over controls. Perfluorocarbons have been commercialized for skincare, as Oxycyte, TherOx, and Cutagenix are each perfluorocarbon emulsions for topical applications.

A hydrogel is a three-dimensional network of hydrophilic polymers that can swell and hold vast amounts of water while maintaining their underlying chemical structure due to physical cross-linking of polymer chains. Cross-linking in hydrogels may be either physical or chemical. Hydrogels are highly absorbent materials that do not dissolve in water. In some embodiments, hydrogels may include chitosan, hyaluronic acid, heparin, alginate, fibrin, PEP, polyvinyl alcohol, polyethylene glycol, sodium polyacrylate, acrylate polymers and copolymers thereof. In some embodiments, hydrogels may be peptide based. The properties of a hydrogel are highly dependent on both the type of cross links that hold the hydrogel together as well as quality and strength of those same cross links.

In some embodiments, physically cross-linked hydrogels may be assembled through the gelation of nanofibrous peptide assemblies and/or through non-covalent interactions of cross-linked domains. In some embodiments, chemically cross-linked hydrogels may be assembled through polymerization reactions, photopolymerization reactions, and/or silanisation reactions.

Perfluorocarbons incorporated into a solid substance may be easier to handle and apply than the substance in liquid form. The Leipzig lab developed a degradable hydrogel containing perfluorocarbon chains which was able to absorb and release oxygen while simultaneously providing a sturdy, easy to handle medium in which to transport the perfluorocarbons.

Membrane electrolytic assemblies are oxygen concentrators. Combining a membrane electrolytic assembly with perfluorocarbon layers allows for the perfluorocarbon layer to continuously store large amounts of oxygen, ready to be released at a wound site. Membrane electrolytic assemblies concentrate gaseous oxygen over the wound space, which then proportionally increases the oxygen concentration within the wound space itself.

Wound exudate has the capability of creating a barrier to oxygen exchange from the environment surrounding the wound space to the wound space itself. This can block the supply of external oxygen from a wound. By utilizing negative pressure wound therapies, or absorption materials capable of wicking away the wound exudate efficiently, oxygen can more effectively be delivered to the wound from the environment surrounding the wound space.

### Method of Treating a Wound

Some preferred embodiments described herein the specification provide a method of treating a wound, intact tissue, or other suitable location. Such a method may include placing one or more perfluorocarbon layers, either separately or by placing a multi-layered wound dressing having one or more perfluorocarbon layers over the wound. The method may comprise adhering the separate one or more perfluorocarbon layers and/or the multi-layer wound dressing having one or more perfluorocarbon layers to healthy skin around the wound, such as the periwound area. The method may further comprise one or more of the following steps: A further wound dressing can be placed over the separate one or more perfluorocarbon layers or multi-layered wound dressing having the one or more perfluorocarbon layers that is placed over the wound. Wound exudate, or any moist or aqueous medium other than wound exudate, may be provided to reach and/or touch the one or more perfluorocarbon layers. Wound exudate, or any moist or aqueous medium other than wound exudate may be diffused or wicked into the wound dressing incorporating the one or more perfluorocarbon layers or into a wound dressing provided over the one or more perfluorocarbon layers. Negative pressure may be applied to the separate one or more perfluorocarbon layers or multi-layered wound dressing having the one or more perfluorocarbon layers, as described in the following “Negative Pressure Wound Therapy (NPWT) Systems” section or described elsewhere herein the specification, such that wound exudate is suctioned into the one or more perfluorocarbon layers directly, or into the wound dressing incorporating the one or more perfluorocarbon layers, or into a wound dressing provided over the one or more perfluorocarbon layers.

The method of treating a wound, intact tissue, or other suitable location as described above or described elsewhere herein may further comprise delivering negative pressure through the wound contact layer to the wound, as described in the following “Negative Pressure Wound Therapy (NPWT) Systems” section or described elsewhere herein the specification. The wound contact layer may substantially maintain the negative pressure delivered for at least about 24 hours, or for at least about 48 hours, or for at least about 72 hours. Alternatively, the method of treating a wound, intact tissue, or other suitable location may comprise applying compression (positive) pressure through the wound contact layer to the wound. Alternatively, the method may comprise altering ambient pressure, negative pressure and compression pressure in a programmable manner through the wound contact layer to the wound.

In embodiments, the method of treating a wound, intact tissue, or other suitable location may comprise using the wound contact layer, or the wound treatment system or wound dressing that comprises the wound contact layer, under ambient conditions not in connection with a negative pressure wound therapy system as described above, or described elsewhere herein.

In some embodiments, a method of treating a wound, intact tissue, or other suitable location may reduce the wound bioburden, for example, at least in vitro, by reducing the numbers (CFU/sample) of viable microorganisms within the first 4 hours after the application wound contact layer. In some examples, the numbers of viable microorganisms may be reduced by four log or more, 48 to 72 hours after positioning the wound dressing in contact with the microorganisms.

### Negative Pressure Wound Therapy (NPWT) Systems

It will be understood that embodiments of the present disclosure are generally applicable to, but not limited to, use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760-X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

The negative pressure range for some embodiments of the present disclosure can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some embodiments, the pressure range can be between about −40 mmHg and −150 mmHg. Alternatively, a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even −150 mmHg, can be supplied by the negative pressure apparatus.

In some embodiments of wound closure devices described herein, increased wound contraction can lead to increased tissue expansion in the surrounding wound tissue. This effect may be increased by varying the force applied to the tissue, for example by varying the negative pressure applied to the wound over time, possibly in conjunction with increased tensile forces applied to the wound via embodiments of the wound closure devices. In some embodiments, negative pressure may be varied over time for example using a sinusoidal wave, square wave, or in synchronization with one or more patient physiological indices (e.g., heartbeat). Examples of such applications where additional disclosure relating to the preceding may be found include U.S. Pat. No. 8,235,955, titled “Wound treatment apparatus and method,” issued on Aug. 7, 2012; and U.S. Pat. No. 7,753,894, titled “Wound cleansing apparatus with stress,” issued Jul. 13, 2010. The disclosures of both of these patents are hereby incorporated by reference in their entirety.

Embodiments of the wound dressings, wound dressing components, wound treatment apparatuses and methods described herein may also be used in combination or in addition to those described in International Application No. PCT/IB2013/001469, filed May 22, 2013, published as WO 2013/175306 A2 on Nov. 28, 2013, titled “APPARATUSES AND METHODS FOR NEGATIVE PRESSURE WOUND THERAPY,” International Application No. PCT/IB2013/002060, filed on Jul. 31, 2013, published as WO2014/020440, entitled “WOUND DRESSING,” the disclosures of which are hereby incorporated by reference in their entireties. Embodiments of the wound dressings, wound treatment apparatuses and methods described herein may also be used in combination or in addition to those described in U.S. Pat. No. 9,061,095, titled “WOUND DRESSING AND METHOD OF USE,” issued on Jun. 23, 2015; and U.S. Application Publication No. 2016/0339158, titled “FLUIDIC CONNECTOR FOR NEGATIVE PRESSURE WOUND THERAPY,” published on Nov. 24, 2016, the disclosures of which are hereby incorporated by reference in its entirety, including further details relating to embodiments of wound dressings, the wound dressing components and principles, and the materials used for the wound dressings.

Additionally, some embodiments related to TNP wound treatment comprising a wound dressing in combination with a pump or associated electronics described herein may also be used in combination or in addition to those described in International Publication No. WO 2016/174048 A1, entitled “REDUCED PRESSURE APPARATUSES”, published on Nov. 3, 2016, the entirety of which is hereby incorporated by reference. In some of these embodiments, the pump or associate electronic components may be integrated into the wound dressing to provide a single article to be applied to the wound.

### Multi-Layered Wound Dressings for NPWT

FIGS. 1A and 1B illustrate an example of a negative pressure wound therapy system 700. The system includes a wound cavity 710 covered by a wound dressing 720, which can be a dressing according to any of the examples described herein. The dressing 720 can be positioned on, inside, over, or around the wound cavity 710 and further seal the wound cavity so that negative pressure can be maintained in the wound cavity. For example, a film layer of the wound dressing 720 can provide substantially fluid impermeable seal over the wound cavity 710. In some embodiments, a wound filler, such as a layer of foam or gauze, may be utilized to pack the wound. The wound filler may include one or more perfluorocarbon layers (e.g. an oxygen releasing layer) as described herein this section or elsewhere in the specification. For example, in a traditional negative pressure wound therapy system utilizing foam or gauze, such as the Smith & Nephew RENASYS Negative Pressure Wound Therapy System utilizing foam (RENASYS-F) or gauze (RENASYS-G), the foam or gauze may be supplemented with perfluorocarbon layers as described above. When supplementing a foam or gauze layer or other wound packing material, the one or more perfluorocarbon layers may either be separately inserted into the wound or may be pre-attached with the wound packing material for insertion into the wound.

A single or multi lumen tube or conduit 740 connects the wound dressing 720 with a negative pressure device 750 configured to supply reduced pressure. The negative pressure device 750 includes a negative pressure source. The negative pressure device 750 can be a canister-less device (meaning that exudate is collected in the wound dressing and/or is transferred via the tube 740 for collection to another location). In some embodiments, the negative pressure device 750 can be configured to include or support a canister. Additionally, in any of the embodiments disclosed herein, the negative pressure device 750 can be fully or partially embedded in, mounted to, or supported by the wound dressing 720.

The conduit 740 can be any suitable article configured to provide at least a substantially sealed fluid flow path or pathway between the negative pressure device 750 and the wound cavity 710 so as to supply reduced pressure to the wound cavity. The conduit 740 can be formed from polyurethane, PVC, nylon, polyethylene, silicone, or any other suitable rigid or flexible material. In some embodiments, the wound dressing 720 can have a port configured to receive an end of the conduit 740. For example, a port can include a hole in the film layer. In some embodiments, the conduit 740 can otherwise pass through and/or under a film layer of the wound dressing 720 to supply reduced pressure to the wound cavity 710 so as to maintain a desired level of reduced pressure in the wound cavity. In some embodiments, at least a part of the conduit 740 is integral with or attached to the wound dressing 720.

FIG. 1B illustrates an embodiment of a negative pressure wound treatment system employing a wound dressing 100 in conjunction with a fluidic connector 110. Additional examples related to negative pressure wound treatment comprising a wound dressing in combination with a pump as described herein may also be used in combination or in addition to those described in U.S. Pat. No. 9,061,095, which is incorporated by reference in its entirety. Here, the fluidic connector 110 may comprise an elongate conduit, more preferably a bridge 120 having a proximal end 130 and a distal end 140, and an applicator 180 at the distal end 140 of the bridge 120. The system may include a source of negative pressure such as a pump or negative pressure unit 150 capable of supplying negative pressure. The pump may comprise a canister or other container for the storage of wound exudates and other fluids that may be removed from the wound. A canister or container may also be provided separate from the pump. In some embodiments, the pump 150 can be a canisterless pump such as the PICO™ pump, as sold by Smith & Nephew. The pump 150 may be connected to the bridge 120 via a tube, or the pump 150 may be connected directly to the bridge 120. In use, the dressing 100 is placed over a suitably-prepared wound, which may in some cases be filled with a wound packing material such as foam or gauze as described above. The applicator 180 of the fluidic connector 110 has a sealing surface that is placed over an aperture in the dressing 100 and is sealed to the top surface of the dressing 100. Either before, during, or after connection of the fluidic connector 110 to the dressing 100, the pump 150 is connected via the tube to the coupling 160, or is connected directly to the bridge 120. The pump is then activated, thereby supplying negative pressure to the wound. Application of negative pressure may be applied until a desired level of healing of the wound is achieved.

As shown in FIG. 1C, the fluidic connector 110 preferably comprises an enlarged distal end, or head 140 that is in fluidic communication with the dressing 100 as will be described in further detail below. In one embodiment, the enlarged distal end has a round or circular shape. The head 140 is illustrated here as being positioned near an edge of the dressing 100, but may also be positioned at any location on the dressing. For example, some embodiments may provide for a centrally or off-centered location not on or near an edge or corner of the dressing 100. In some embodiments, the dressing 100 may comprise two or more fluidic connectors 110, each comprising one or more heads 140, in fluidic communication therewith. In a preferred embodiment, the head 140 may measure 30 mm along its widest edge. The head 140 forms at least in part the applicator 180, described above, that is configured to seal against a top surface of the wound dressing.

FIG. 1D illustrates a cross-section through a wound dressing 100 similar to the wound dressing 10 as described in International Patent Publication WO2013175306 A2, which is incorporated by reference in its entirety, along with fluidic connector 110. The wound dressing 100, which can alternatively be any wound dressing embodiment disclosed herein or any combination of features of any number of wound dressing embodiments disclosed herein, can be located over a wound site to be treated. The dressing 100 may be placed as to form a sealed cavity over the wound site. In a preferred embodiment, the dressing 100 comprises a top or cover layer, or backing layer 220 attached to an optional wound contact layer 222, both of which are described in greater detail below. These two layers 220, 222 are preferably joined or sealed together so as to define an interior space or chamber. This interior space or chamber may comprise additional structures that may be adapted to distribute or transmit negative pressure, store wound exudate and other fluids removed from the wound, and other functions which will be explained in greater detail below. Examples of such structures, described below, include a transmission layer 226 and an absorbent layer 221.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound.

As illustrated in FIG. 1D, the wound contact layer 222 can be a polyurethane layer or polyethylene layer or other flexible layer which is perforated, for example via a hot pin process, laser ablation process, ultrasound process or in some other way or otherwise made permeable to liquid and gas. The wound contact layer 222 has a lower surface 224 and an upper surface 223. The perforations 225 preferably comprise through holes in the wound contact layer 222 which enable fluid to flow through the layer 222. The wound contact layer 222 helps prevent tissue ingrowth into the other material of the wound dressing. Preferably, the perforations are small enough to meet this requirement while still allowing fluid to flow therethrough. For example, perforations formed as slits or holes having a size ranging from 0.025 mm to 1.2 mm are considered small enough to help prevent tissue ingrowth into the wound dressing while allowing wound exudate to flow into the dressing. In some configurations, the wound contact layer 222 may help maintain the integrity of the entire dressing 100 while also creating an air-tight seal around the absorbent pad in order to maintain negative pressure at the wound.

Some embodiments of the wound contact layer 222 may also act as a carrier for an optional lower and upper adhesive layer (not shown). For example, a lower pressure sensitive adhesive may be provided on the lower surface 224 of the wound dressing 100 whilst an upper pressure sensitive adhesive layer may be provided on the upper surface 223 of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized may be helpful to adhere the wound dressing 100 to the skin around a wound site. In some embodiments, the wound contact layer may comprise perforated polyurethane film. The lower surface of the film may be provided with a silicone pressure sensitive adhesive and the upper surface may be provided with an acrylic pressure sensitive adhesive, which may help the dressing maintain its integrity. In some embodiments, a polyurethane film layer may be provided with an adhesive layer on both its upper surface and lower surface, and all three layers may be perforated together.

A transmission layer 226 can be located above the wound contact layer 222. In some embodiments, the transmission layer can be a porous material. As used herein the transmission layer can be referred to as a spacer layer and the terms can be used interchangeably to refer to the same component described herein. This transmission layer 226 allows transmission of fluid including liquid and gas away from a wound site into upper layers of the wound dressing. In particular, the transmission layer 226 preferably ensures that an open-air channel can be maintained to communicate negative pressure over the wound area even when the absorbent layer has absorbed substantial amounts of exudates. The layer 226 should preferably remain open under the typical pressures that will be applied during negative pressure wound therapy as described above, so that the whole wound site sees an equalized negative pressure. The layer 226 may be formed of a material having a three-dimensional structure. For example, a knitted or woven spacer fabric (for example Baltex 7970 weft knitted polyester) or a non-woven fabric could be used. The three-dimensional material can comprise a 3D spacer fabric material similar to the material described in International Publication WO 2013/175306 A2 and International Publication WO2014/020440, the disclosures of which are incorporated by reference in their entireties.

In certain embodiments, the wound dressing 100 may incorporate or comprise one or more perfluorocarbon layers as described herein this section or elsewhere in the specification. One of skill in the art will understand that the wound dressing 100 may incorporate any of the one or more perfluorocarbon layers disclosed herein this section or elsewhere in the specification. One of skill in the art will also understand that the one or more perfluorocarbon layers may be incorporated as a whole component layer or a part of a component layer. In some embodiments, the one or more perfluorocarbon layers may be provided below the transmission layer 226. In some embodiments, the one or more perfluorocarbon layers may be provided above the wound contact layer 222. In certain embodiments, the one or more perfluorocarbon layers may replace the transmission layer 226, such that the one or more perfluorocarbon layers are provided between an absorbent layer 221 (described further below) and the wound contact layer 222. In some embodiments, the one or more perfluorocarbon layers can supplement or replace the absorbent layer 221. In some embodiments, the wound dressing 100 does not have the wound contact layer 222, and the one or more perfluorocarbon layers may be the lowermost layer of the wound dressing 100. The one or more perfluorocarbon layers may have same or substantially similar size and shape with the transmission layer 226 and/or the absorbent layer 221.

The one or more perfluorocarbon layers may be constructed to be flexible but stiff enough to withstand negative pressure, such that the one or more perfluorocarbon layers is not collapsed excessively and thereby may transmit negative pressure sufficiently to the wound when negative pressure is supplied to the wound dressing 100. The one or more perfluorocarbon layers may be constructed to include sufficient number or size of pores to enable transmission of negative pressure. The one or more perfluorocarbon layers may include an aperture or hole, for example, under the port, to transmit negative pressure and/or wound fluid. Further, the one or more perfluorocarbon layers may have suitable thickness(es) to transmit suitable negative pressure to the wound. For example, the one or more perfluorocarbon layers may have a thickness of about 1 mm to 10 mm, or 1 mm to 7 mm, or 1.5 mm to 7 mm, or 1.5 mm to 4 mm, or 2 mm to 3 mm. In some embodiments, the one or more perfluorocarbon layers may have a thickness of approximately 2 mm.

In some embodiments, the layer 221 of absorbent material is provided above the transmission layer 226. The absorbent material, which can comprise a foam or non-woven natural or synthetic material, and which may optionally comprise a super-absorbent material, forms a reservoir for fluid, particularly liquid, removed from the wound site. In some embodiments, the layer 221 may also aid in drawing fluids towards the backing layer 220.

The material of the absorbent layer 221 may also prevent liquid collected in the wound dressing 100 from flowing freely within the dressing, and preferably acts so as to contain any liquid collected within the dressing. The absorbent layer 221 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 221 may typically be manufactured from ALLEVYN™ foam, Freudenberg 114-224-4 or Chem-Posite™ 11C-450. In some embodiments, the absorbent layer 221 may comprise a composite comprising superabsorbent powder, fibrous material such as cellulose, and bonding fibers. In a preferred embodiment, the composite is an air-laid, thermally-bonded composite.

In some embodiments, the absorbent layer 221 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. The wicking action also assists in bringing liquid into contact with the upper cover layer to aid increase transpiration rates of the dressing.

An aperture, hole, or orifice 227 is preferably provided in the backing layer 220 to allow a negative pressure to be applied to the dressing 100. The fluidic connector 110 is preferably attached or sealed to the top of the backing layer 220 over the orifice 227 made into the dressing 100, and communicates negative pressure through the orifice 227. A length of tubing may be coupled at a first end to the fluidic connector 110 and at a second end to a pump unit (not shown) to allow fluids to be pumped out of the dressing. Where the fluidic connector is adhered to the top layer of the wound dressing, a length of tubing may be coupled at a first end of the fluidic connector such that the tubing, or conduit, extends away from the fluidic connector parallel or substantially to the top surface of the dressing. The fluidic connector 110 may be adhered and sealed to the backing layer 220 using an adhesive such as an acrylic, cyanoacrylate, epoxy, UV curable or hot melt adhesive. The fluidic connector 110 may be formed from a soft polymer, for example a polyethylene, a polyvinyl chloride, a silicone or polyurethane having a hardness of 30 to 90 on the Shore A scale. In some embodiments, the fluidic connector 110 may be made from a soft or conformable material.

Optionally, the absorbent layer 221 includes at least one through hole 228 located so as to underlie the fluidic connector 110. The through hole 228 may in some embodiments be the same size as the opening 227 in the backing layer, or may be bigger or smaller. As illustrated in FIG. 1D a single through hole can be used to produce an opening underlying the fluidic connector 110. It will be appreciated that multiple openings could alternatively be utilized. Additionally, should more than one port be utilized according to certain embodiments of the present disclosure one or multiple openings may be made in the absorbent layer in registration with each respective fluidic connector. Although not essential to certain embodiments of the present disclosure the use of through holes in the super-absorbent layer may provide a fluid flow pathway which remains unblocked in particular when the absorbent layer is near saturation.

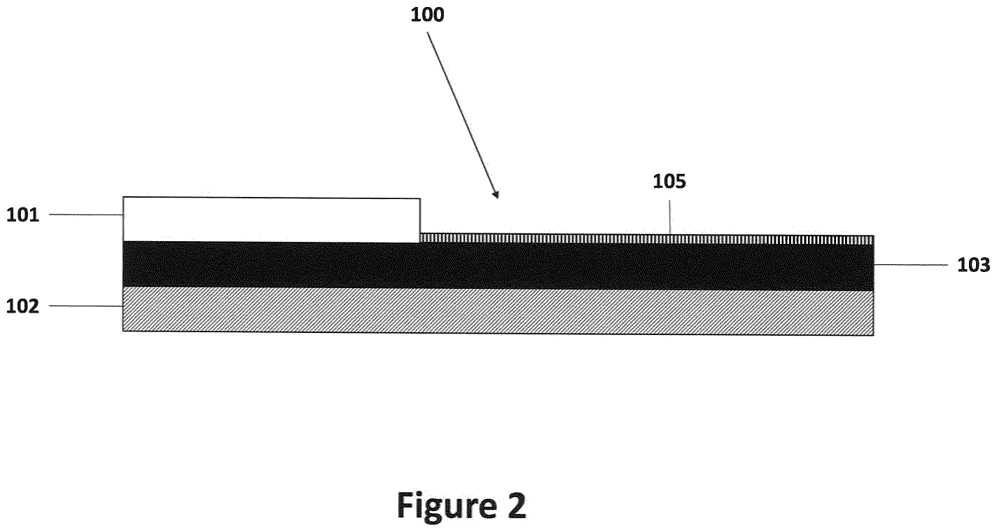

The aperture or through-hole 228 is preferably provided in the absorbent layer 221 beneath the orifice 227 such that the orifice is connected directly to the transmission layer 226 as illustrated in FIG. 1D. This allows the negative pressure applied to the fluidic connector 110 to be communicated to the transmission layer 226 without passing through the absorbent layer 221. This ensures that the negative pressure applied to the wound site is not inhibited by the absorbent layer as it absorbs wound exudates. In other embodiments, no aperture may be provided in the absorbent layer 221, or alternatively a plurality of apertures underlying the orifice 227 may be provided. In further alternative embodiments, additional layers such as another transmission layer or an obscuring layer such as described with reference to FIGS. 2-6 and in International Patent Publication WO2014/020440, the entirety of which is hereby incorporated by reference, may be provided over the absorbent layer 221 and beneath the backing layer 220.

The backing layer 220 is preferably gas impermeable, but moisture vapor permeable, and can extend across the width of the wound dressing 100. The backing layer 220, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way, an effective chamber is made between the backing layer 220 and a wound site where a negative pressure can be established. The backing layer 220 is preferably sealed to the wound contact layer 222 in a border region around the circumference of the dressing, ensuring that no air is drawn in through the border area, for example via adhesive or welding techniques. The backing layer 220 protects the wound from external bacterial contamination (bacterial barrier) and allows liquid from wound exudates to be transferred through the layer and evaporated from the film outer surface. The backing layer 220 preferably comprises two layers; a polyurethane film and an adhesive pattern spread onto the film. The polyurethane film is preferably moisture vapor permeable and may be manufactured from a material that has an increased water transmission rate when wet. In some embodiments, the moisture vapor permeability of the backing layer increases when the backing layer becomes wet. The moisture vapor permeability of the wet backing layer may be up to about ten times more than the moisture vapor permeability of the dry backing layer.

The absorbent layer 221 may be of a greater area than the transmission layer 226, such that the absorbent layer overlaps the edges of the transmission layer 226, thereby ensuring that the transmission layer does not contact the backing layer 220. This provides an outer channel of the absorbent layer 221 that is in direct contact with the wound contact layer 222, which aids more rapid absorption of exudates to the absorbent layer. Furthermore, this outer channel ensures that no liquid is able to pool around the circumference of the wound cavity, which may otherwise seep through the seal around the perimeter of the dressing leading to the formation of leaks. As illustrated in FIG. 1D, the absorbent layer 221 may define a smaller perimeter than that of the backing layer 220, such that a boundary or border region is defined between the edge of the absorbent layer 221 and the edge of the backing layer 220.

As shown in FIG. 1D, one embodiment of the wound dressing 100 comprises an aperture 228 in the absorbent layer 221 situated underneath the fluidic connector 110. In use, for example when negative pressure is applied to the dressing 100, a wound facing portion of the fluidic connector may thus come into contact with the transmission layer 226, which can thus aid in transmitting negative pressure to the wound site even when the absorbent layer 221 is filled with wound fluids. Some embodiments may have the backing layer 220 be at least partly adhered to the transmission layer 226. In some embodiments, the aperture 228 is at least 1-2 mm larger than the diameter of the wound facing portion of the fluidic connector 11, or the orifice 227.

In particular for embodiments with a single fluidic connector 110 and through hole, it may be preferable for the fluidic connector 110 and through hole to be located in an off-center position as illustrated in FIG. 1C. Such a location may permit the dressing 100 to be positioned onto a patient such that the fluidic connector 110 is raised in relation to the remainder of the dressing 100. So positioned, the fluidic connector 110 and the filter 214 may be less likely to come into contact with wound fluids that could prematurely occlude the filter 214 so as to impair the transmission of negative pressure to the wound site.

Similar to the embodiments of wound dressings described above, some wound dressings comprise a perforated wound contact layer with silicone adhesive on the skin-contact face and acrylic adhesive on the reverse. In some embodiments, the wound contact layer may be constructed from polyurethane, polyethylene or polyester. Above this bordered layer sits a transmission layer. Above the transmission layer, sits an absorbent layer. The absorbent layer can include a superabsorbent non-woven (NW) pad. The absorbent layer can over-border the transmission layer by approximately 5 mm at the perimeter. The absorbent layer can have an aperture or through-hole toward one end. The aperture can be about 10 mm in diameter. Over the transmission layer and absorbent layer lies a backing layer. The backing layer can be a high moisture vapor transmission rate (MVTR) film, pattern coated with acrylic adhesive. The high MVTR film and wound contact layer encapsulate the transmission layer and absorbent layer, creating a perimeter border of approximately 20 mm. The backing layer can have a 10 mm aperture that overlies the aperture in the absorbent layer. Above the hole can be bonded a fluidic connector that comprises a liquid-impermeable, gas-permeable semi-permeable membrane (SPM) or filter that overlies the aforementioned apertures.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound.

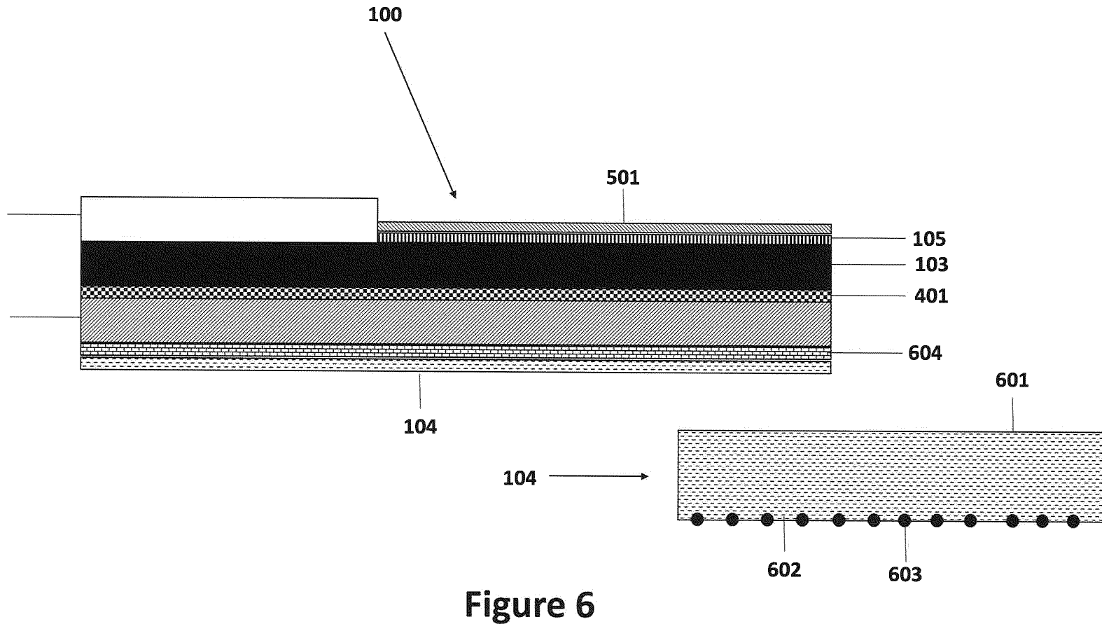

As illustrated in FIGS. 2-6, in embodiments, the wound contact layer 104 can be a polyurethane layer or polyethylene layer or other flexible layer which is perforated, for example via a hot pin process, laser ablation process, ultrasound process or in some other way or otherwise made permeable to liquid and gas. The wound contact layer 104 has a lower surface 602 and an upper surface 601 as shown in FIG. 6. The perforations 603 preferably comprise through holes in the wound contact layer 104 which enable fluid to flow through the layer 104. The wound contact layer 104 helps prevent tissue ingrowth into the other material of the wound dressing. Preferably, the perforations are small enough to meet this requirement while still allowing fluid to flow therethrough. For example, perforations formed as slits or holes having a size ranging from 0.025 mm to 1.2 mm are considered small enough to help prevent tissue ingrowth into the wound dressing while allowing wound exudate to flow into the dressing. In some configurations, the wound contact layer 104 may help maintain the integrity of the entire dressing 100 while also creating an air-tight seal around the absorbent pad in order to maintain negative pressure at the wound.

Some embodiments of the wound contact layer 104 may also act as a carrier for an optional lower and upper adhesive layer (not shown). For example, a lower pressure sensitive adhesive may be provided on the lower surface 602 of the wound dressing 100 whilst an upper pressure sensitive adhesive layer may be provided on the upper surface 601 of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized may be helpful to adhere the wound dressing 100 to the skin around a wound site. In some embodiments, the wound contact layer may comprise perforated polyurethane film. The lower surface of the film may be provided with a silicone pressure sensitive adhesive and the upper surface may be provided with an acrylic pressure sensitive adhesive, which may help the dressing maintain its integrity. In some embodiments, a polyurethane film layer may be provided with an adhesive layer on both its upper surface and lower surface, and all three layers may be perforated together.

A transmission layer 604 can be located above the wound contact layer 104 as shown in FIG. 6. In some embodiments, the transmission layer can be a porous material. As used herein the transmission layer can be referred to as a spacer layer and the terms can be used interchangeably to refer to the same component described herein. This transmission layer 604 allows transmission of fluid including liquid and gas away from a wound site into upper layers of the wound dressing. In particular, the transmission layer 604 preferably ensures that an open-air channel can be maintained to communicate negative pressure over the wound area even when the absorbent layer has absorbed substantial amounts of exudates. The layer 604 should preferably remain open under the typical pressures that will be applied during negative pressure wound therapy as described above, so that the whole wound site sees an equalized negative pressure. The layer 604 may be formed of a material having a three-dimensional structure. For example, a knitted or woven spacer fabric (for example Baltex 7970 weft knitted polyester) or a non-woven fabric could be used. The three-dimensional material can comprise a 3D spacer fabric material similar to the material described in International Publication WO 2013/175306 A2 and International Publication WO2014/020440, the disclosures of which are incorporated by reference in their entireties.

In certain embodiments, the wound dressing 100 may incorporate or comprise one or more perfluorocarbon layers as described herein this section or elsewhere in the specification. One of skill in the art will understand that the wound dressing 100 may incorporate any of the one or more perfluorocarbon layers disclosed herein this section or elsewhere in the specification. One of skill in the art will also understand that the one or more perfluorocarbon layers may be incorporated as a whole component layer or a part of a component layer. In some embodiments, the one or more perfluorocarbon layers may be provided below the transmission layer 604. In some embodiments, the one or more perfluorocarbon layers may be provided above the wound contact layer 104. In certain embodiments, the one or more perfluorocarbon layers may replace the transmission layer 604, such that the one or more perfluorocarbon layers are provided between an absorbent layer 401 (described further below) and the wound contact layer 104. In some embodiments, the one or more perfluorocarbon layers can supplement or replace the absorbent layer 401. In some embodiments, the wound dressing 100 does not have the wound contact layer 104, and the one or more perfluorocarbon layers may be the lowermost layer of the wound dressing 100. The one or more perfluorocarbon layers may have same or substantially similar size and shape with the transmission layer 604 and/or the absorbent layer 401.

The one or more perfluorocarbon layers may be constructed to be flexible but stiff enough to withstand negative pressure, such that the one or more perfluorocarbon layers is not collapsed excessively and thereby may transmit negative pressure sufficiently to the wound when negative pressure is supplied to the wound dressing 100. The one or more perfluorocarbon layers may be constructed to include sufficient number or size of pores to enable transmission of negative pressure. The one or more perfluorocarbon layer may include an aperture or hole, for example, under the port, to transmit negative pressure and/or wound fluid. Further, the one or more perfluorocarbon layers may have suitable thickness(es) to transmit suitable negative pressure to the wound. For example, the one or more perfluorocarbon layers may have a thickness of about 1 mm to 10 mm, or 1 mm to 7 mm, or 1.5 mm to 7 mm, or 1.5 mm to 4 mm, or 2 mm to 3 mm. In some embodiments, the one or more perfluorocarbon layers may have a thickness of approximately 2 mm.

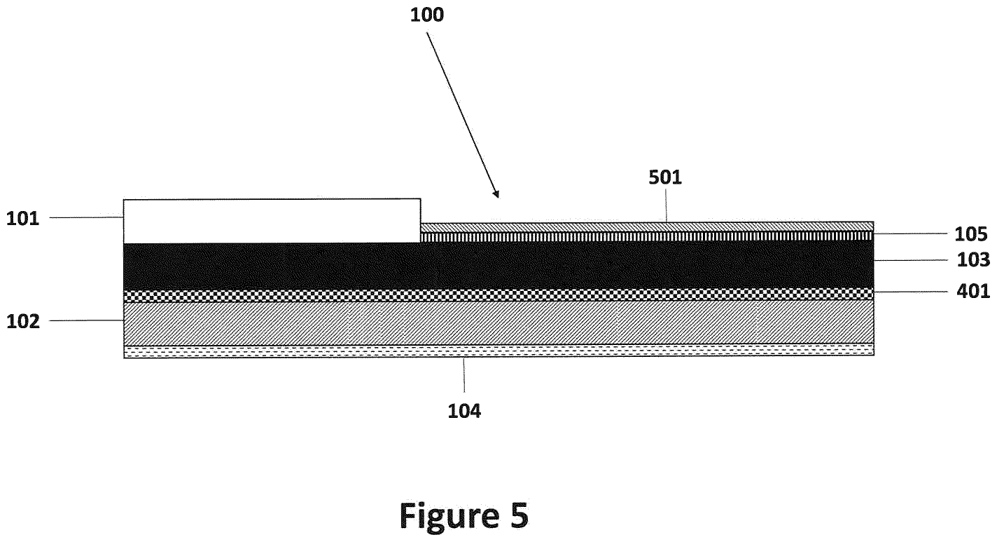

In some embodiments, the layer 401 of absorbent material is provided above the transmission layer 604. The absorbent material, which can comprise a foam or non-woven natural or synthetic material, and which may optionally comprise a super-absorbent material, forms a reservoir for fluid, particularly liquid, removed from the wound site. In some embodiments, the layer 401 may also aid in drawing fluids towards the cover layer 105 as shown in FIG. 5.

The material of the absorbent layer 401 may also prevent liquid collected in the wound dressing 100 from flowing freely within the dressing, and preferably acts so as to contain any liquid collected within the dressing. The absorbent layer 401 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 401 may typically be manufactured from ALLEVYN™ foam, Freudenberg 114-224-4 or Chem-Posite™ 11C-450. In some embodiments, the absorbent layer 401 may comprise a composite comprising superabsorbent powder, fibrous material such as cellulose, and bonding fibers. In a preferred embodiment, the composite is an air-laid, thermally-bonded composite.

In some embodiments, the absorbent layer 401 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. The wicking action also assists in bringing liquid into contact with the upper cover layer to aid increase transpiration rates of the dressing.

The cover layer 105 is preferably gas impermeable, but moisture vapor permeable, and can extend across the width of the wound dressing 100. The cover layer 105, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way, an effective chamber is made between the cover layer 105 and a wound site where a negative pressure can be established. The cover layer 105 is preferably sealed to the wound contact layer 104 in a border region around the circumference of the dressing, ensuring that no air is drawn in through the border area, for example via adhesive or welding techniques. The cover layer 105 protects the wound from external bacterial contamination (bacterial barrier) and allows liquid from wound exudates to be transferred through the layer and evaporated from the film outer surface. The cover layer 105 preferably comprises two layers; a polyurethane film and an adhesive pattern spread onto the film. The polyurethane film is preferably moisture vapor permeable and may be manufactured from a material that has an increased water transmission rate when wet. In some embodiments, the moisture vapor permeability of the backing layer increases when the backing layer becomes wet. The moisture vapor permeability of the wet backing layer may be up to about ten times more than the moisture vapor permeability of the dry backing layer.

The absorbent layer 401 may be of a greater area than the transmission layer 604, such that the absorbent layer overlaps the edges of the transmission layer 604, thereby ensuring that the transmission layer does not contact the cover layer 105. This provides an outer channel of the absorbent layer 401 that is in direct contact with the wound contact layer 104, which aids more rapid absorption of exudates to the absorbent layer. Furthermore, this outer channel ensures that no liquid is able to pool around the circumference of the wound cavity, which may otherwise seep through the seal around the perimeter of the dressing leading to the formation of leaks. As illustrated in FIG. 2C, the absorbent layer 604 may define a smaller perimeter than that of the cover layer 105, such that a boundary or border region is defined between the edge of the absorbent layer 401 and the edge of the cover layer 105.

In some embodiments, the one or more perfluorocarbon layers 102 may incorporate nonafluorohexyltriethoxysilane. In some embodiments, the one or more perfluorocarbon layers 102 may incorporate pentafluorophenyltriethoxysilane. The one or more perfluorocarbon layers 102 may incorporate 1H, 1H, 2H, 2H-perfluorodecyltriethoxysilane. The one or more perfluorocarbon layers 102 may incorporate 1H, 1H, 2H, 2H-perfluorooctyltriethoxysilane.

The absorbent layer 401 may have a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

Similar to the embodiments of wound dressings described above, some wound dressings comprise a perforated wound contact layer with silicone adhesive on the skin-contact face and acrylic adhesive on the reverse. In some embodiments, the wound contact layer may be constructed from polyurethane, polyethylene or polyester. Above this bordered layer sits a transmission layer. Above the transmission layer, sits an absorbent layer. The absorbent layer can include a superabsorbent non-woven (NW) pad. The absorbent layer can over-border the transmission layer by approximately 5 mm at the perimeter. The absorbent layer can have an aperture or through-hole toward one end. The aperture can be about 10 mm in diameter. Over the transmission layer and absorbent layer lies a backing layer. The cover layer 105 can be a high moisture vapor transmission rate (MVTR) film, pattern coated with acrylic adhesive. The high MVTR film and wound contact layer encapsulate the transmission layer and absorbent layer, creating a perimeter border of approximately 20 mm. The backing layer can have a 10 mm aperture that overlies the aperture in the absorbent layer. Above the hole can be bonded a fluidic connector that comprises a liquid-impermeable, gas-permeable semi-permeable membrane (SPM) or filter that overlies the aforementioned apertures.

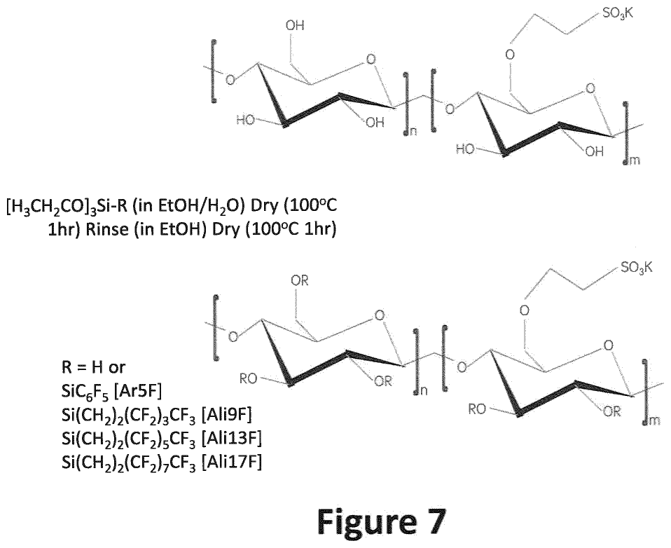

As described above and shown in FIG. 7, in certain examples, perfluorocarbon may be bound to cellulose fibers, such as DURAFIBER via a silanisation process to form a perfluorocarbon layer such as described elsewhere herein. Here, one or more layers 103, such as a cellulose dressing layer such as a DURAFIBER layer, may contain one or more primary, secondary, or tertiary alcohol functional groups, as shown in the scheme in FIG. 7. In some embodiments, the dressing layer may be reacted with (H₃CH₂CO)₃Si—R in an ethanol and water mixture, followed by a drying step at 100° C. for one hour, followed by an ethanol rinse, followed by a final drying step at 100° C. for one hour, where R represents either H, SiC₆F₅[Ar₅F], Si(CH₂)₂(CF₂)₃CF₃ [Ali9F], Si(CH₂)₂(CF₂)₅CF₃ [Ali13F],or Si(CH₂)₂(CF₂)₇CF₃ [Ali17F]. In some embodiments, the layer may be reacted with Cl—R in acetone and refluxed at 80°° C., where R would represent either SiC₆F₅[Ar5F], Si(CH₂)₂(CF₂)₃CF₃ [Ali9F], Si(CH₂)₂(CF₂)₅CF₃ [Ali13F], or Si(CH₂)₂(CF₂)₇CF₃ [Ali17F]. In some embodiments, the layer may be reacted with Cl₃—R in acetone and refluxed at 80° C., followed by an addition of water in a hydrolysis and condensation step, where R may represent either SiC₆F₅[Ar5F], Si(CH₂)₂(CF₂)₃CF₃ [Ali9F], Si(CH₂)₂(CF₂)₅CF₃ [Ali13F],or Si(CH₂)₂(CF₂)₇CF₃ [Ali17F]. In some embodiments, the layer may be reacted with Cl₃—R in acetone, refluxed at 80° C., followed by an addition of water, where R would represent either SiC₆F₅[Ar5F], Si(CH₂)₂(CF₂)₃CF₃ [Ali9F], Si(CH₂)₂(CF₂)₅CF₃ [Ali13F],or Si(CH₂)₂(CF₂)₇CF₃ [Ali17F].

### Multi-Layered Dressing for Use Without Negative Pressure

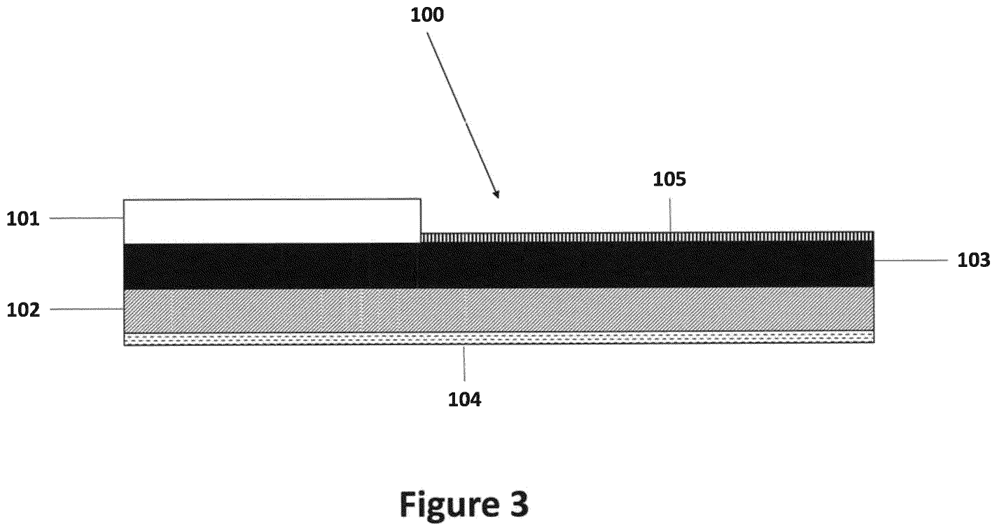

FIGS. 2-6 illustrate various embodiments of a wound dressing 100 that can be used for healing a wound without negative pressure. FIG. 2 illustrates a cross-section of the perfluorocarbon wound dressing, and FIG. 3 shows each of the separate component layers of the perfluorocarbon wound dressing. As shown in the dressing of FIGS. 2 and 3, the wound dressings can have multiple layers. The wound dressing 100 of FIGS. 2 and 3 include a membrane electrolytic apparatus 101, one or more perfluorocarbon layers 102, and one or more dressing layers 103. The wound dressing 100 can also include a cover layer 105 and an optional wound contact layer 104 as described herein. In some embodiments, the cover layer 105 may be permeable to moisture and/or air. The wound dressing can include various layers positioned between the wound contact layer 104 and cover layer 105. For example, the dressing can include one or more absorbent layers or one or more transmission layers as described herein with reference to FIGS. 2-6.

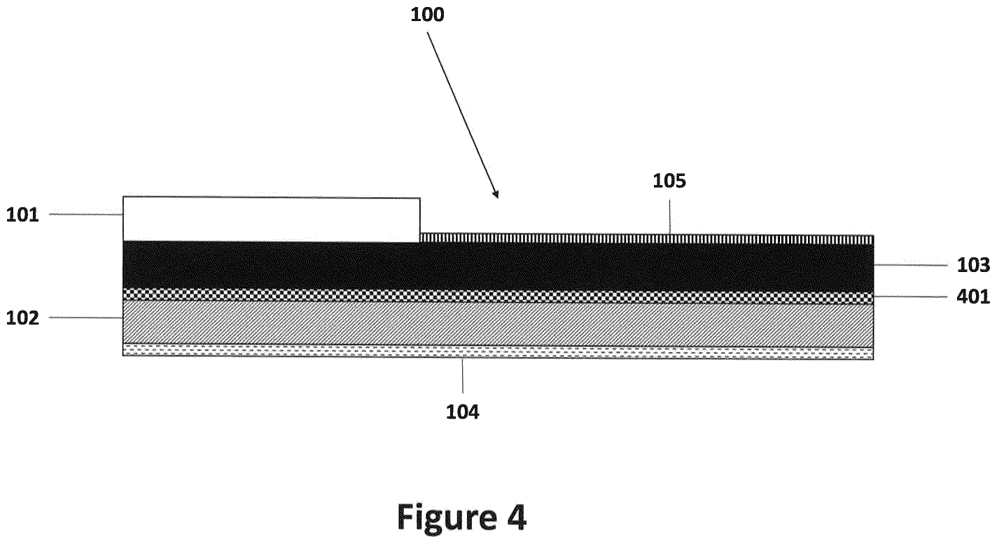

As shown in FIGS. 2-6, the dressing 100 may include a perforated wound contact layer 104 and a top film 105. Further components of the wound dressing 100 include a foam layer, such as a layer of polyurethane hydrocellular foam, of a suitable size to cover the recommended dimension of wounds corresponding to the particular dressing size chosen. An optional layer of activated charcoal cloth (not shown) of similar or slightly smaller dimensions than layer 104 may be provided to allow for odor control. An absorbent layer 401, such as a layer of superabsorbent air-laid material containing cellulose fibres and a superabsorbent polyacrylate particulates, may be provided over layer 104, of dimensions slightly larger than layer 104, and allows for an overlap of superabsorbent material and acts as leak prevention, as shown in FIG. 4. A masking or obscuring layer 501, such as a layer of three-dimensional knitted spacer fabric, may be provided over layer 105, providing protection from pressure, while allowing partial masking of the top surface of the superabsorber where coloured exudate would remain, as shown in FIG. 5. In this embodiment this is of smaller dimension (in plain view) than the layer 105, to allow for visibility of the edge of the absorbent layer, which can be used by clinicians to assess whether the dressing needs to be changed.

The wound dressing 100 may incorporate or comprise one or more perfluorocarbon layers as described herein this section or elsewhere. One of skill in the art will understand that the wound dressing 100 may incorporate any of the one or more perfluorocarbon layers disclosed herein this section or elsewhere in the specification. One of skill in the art will also understand that the one or more perfluorocarbon layers may be incorporated as a whole component layer or a part of a component layer. In some embodiments, the perfluorocarbon layers may be provided below the cover layer 105. In some embodiments, the perfluorocarbon layers may be provided above the wound contact layer 104. In certain embodiments, the dressing 100 may not include the wound contact layer 104, such that one of the perfluorocarbon layers may be the lowermost layer and be configured to touch the wound surface. In some embodiments, the perfluorocarbon layers may be provided below an optional foam layer. In some embodiments, the dressing 100 may include only the cover layer 105, a membrane electrolytic apparatus 101, one or more dressing layers 103, and the one or more perfluorocarbon layers 102.

As described herein, the one or more perfluorocarbon layers, may be incorporated into or used with commercially available dressings, such as DURAFIBER, ALLEVYN™ foam, ALLEVYN™ Life, ALLEVYN™ Adhesive, ALLEVYN™ Gentle Border, ALLEVYN™ Gentle, ALLEVYN™ Ag Gentle Border, ALLEVYN™ Ag Gentle, Opsite Post-Op Visible, and Aquacell. In some embodiments, the wound dressing 100 may include the cover layer 105, the wound contact layer 104, and the perfluorocarbon layers sandwiched therebetween, similarly with the wound dressing format described previously herein relation to FIGS. 2-6. In some embodiments, the wound dressing 500 may include the cover layer 105, an absorbent layer 401, the perfluorocarbon layers below the absorbent layer 401, and the wound contact layer 104, similarly with the wound dressing format described previously herein relation to FIG. 4.

In some embodiments, the one or more perfluorocarbon layers 102 may incorporate nonafluorohexyltriethoxysilane. In some embodiments, the one or more perfluorocarbon layers 102 may incorporate pentafluorophenyltriethoxysilane. The one or more perfluorocarbon layers 102 may incorporate 1H, 1H, 2H, 2H-perfluorodecyltriethoxysilane. The one or more perfluorocarbon layers 102 may incorporate 1H, 1H, 2H, 2H-perfluorooctyltriethoxysilane.

The absorbent layer 401 may have a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

Further details regarding wound dressings that may be combined with or be used in addition to the embodiments described herein, are found in U.S. Pat. No. 9,877,872, issued on Jan. 30, 2018, titled “WOUND DRESSING AND METHOD OF TREATMENT,” the disclosure of which are hereby incorporated by reference in its entirety, including further details relating to embodiments of wound dressings, the wound dressing components and principles, and the materials used for the wound dressings.

### Perfluorocarbon Layer Oxygen Uptake and Release

In some embodiments, the wound dressing 100 contains one or more perfluorocarbon layers 102. In some embodiments, the one or more layers 102 incorporate pentafluorophenyltriethoxysilane. In some embodiments, the one or more layers 102 incorporate nonafluorohexyltriethoxysilane. In some embodiments, the one or more layers 102 incorporate 1H, 1H, 2H, 2H-perfluorodecyltriethoxysilane. In some embodiments, the one or more layers 102 incorporate 1H, 1H, 2H, 2H-perfluorooctyltriethoxysilane.

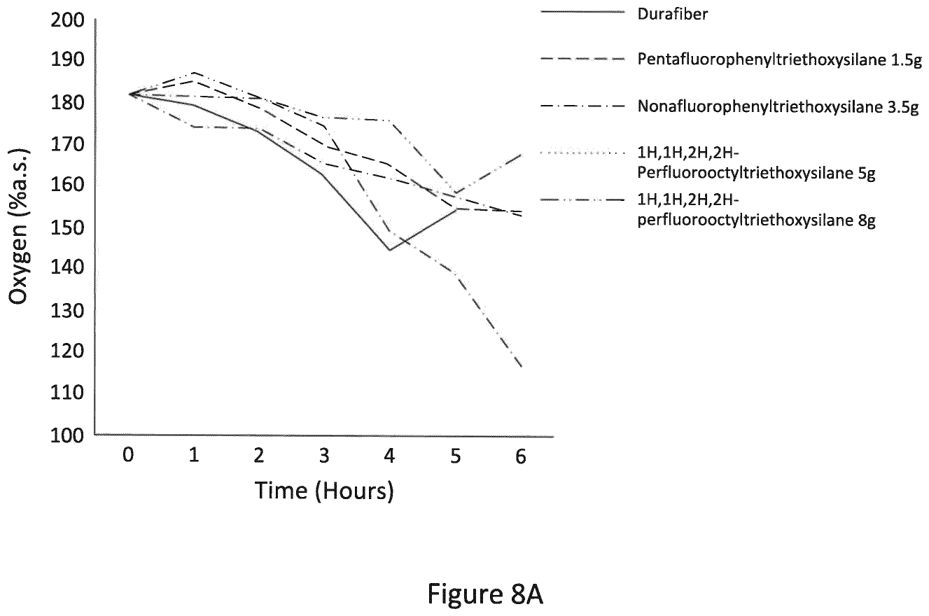

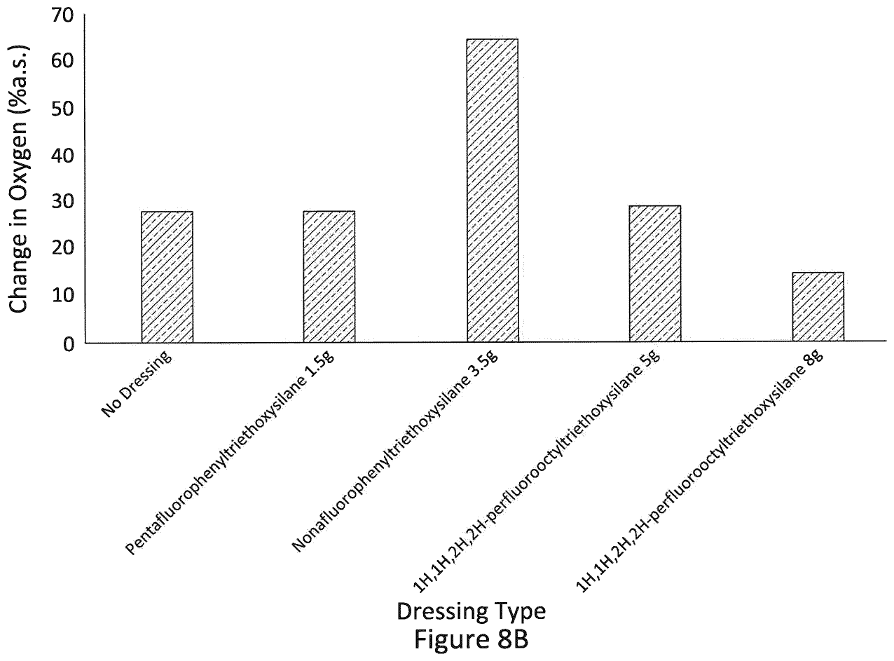

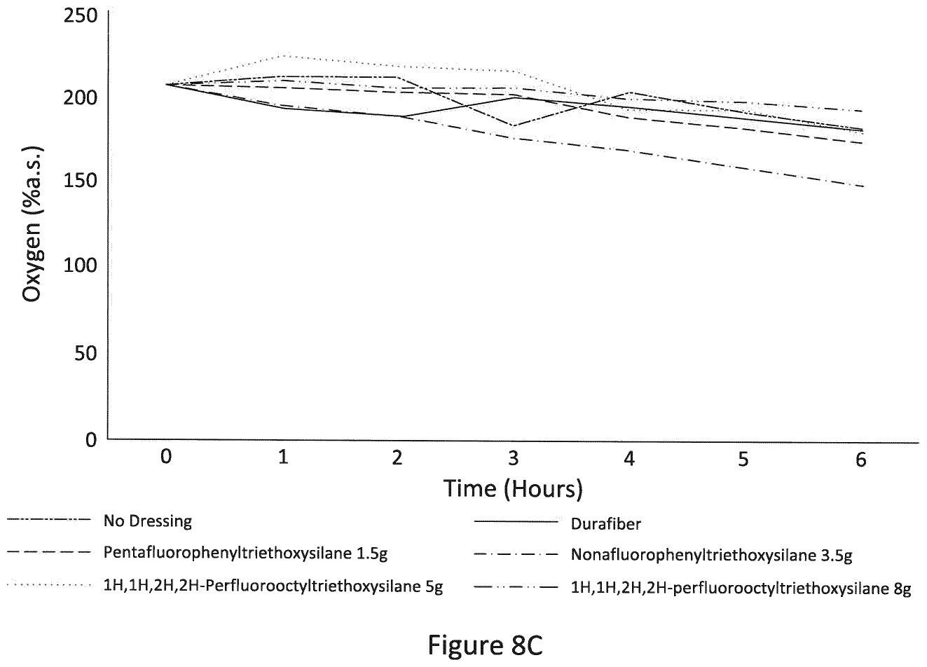

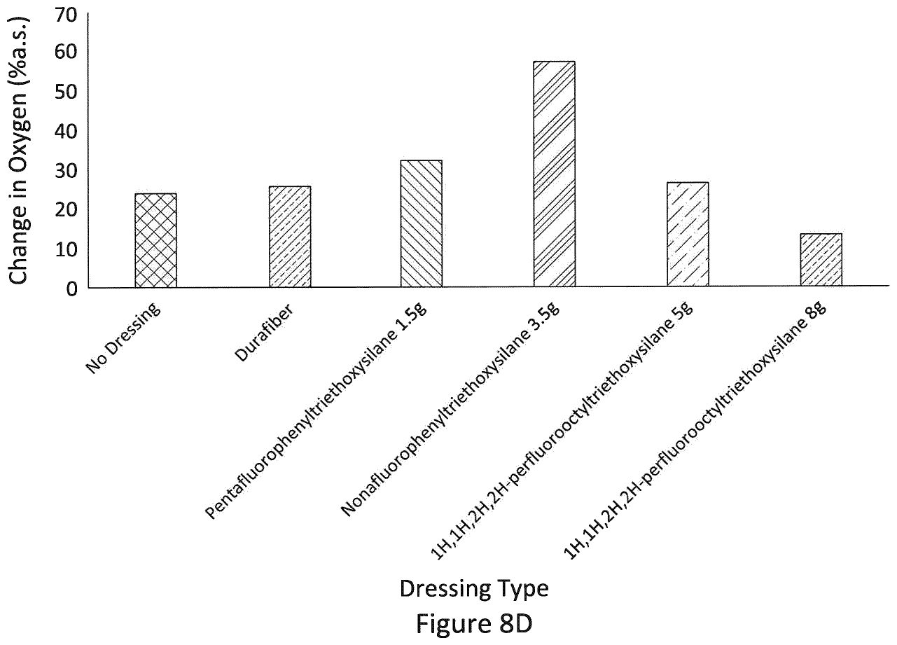

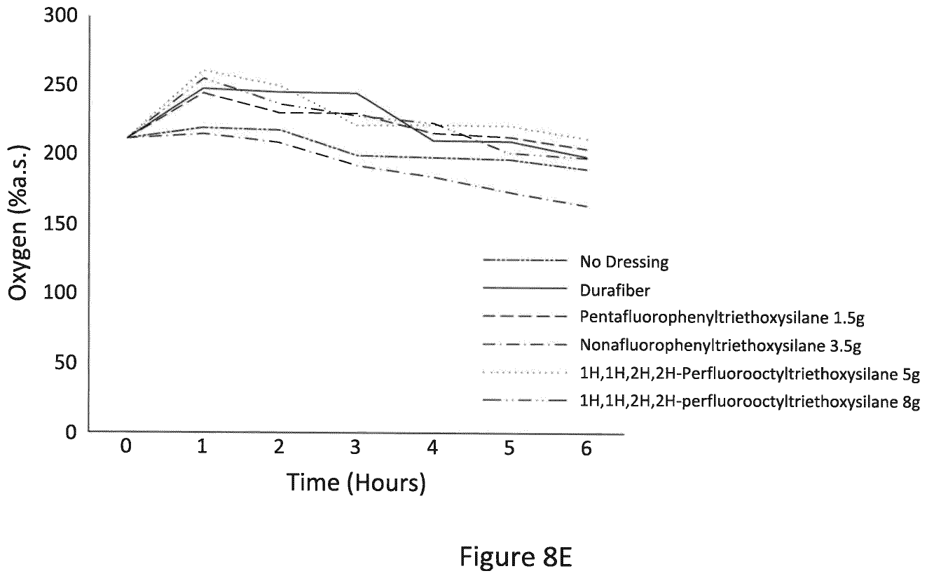

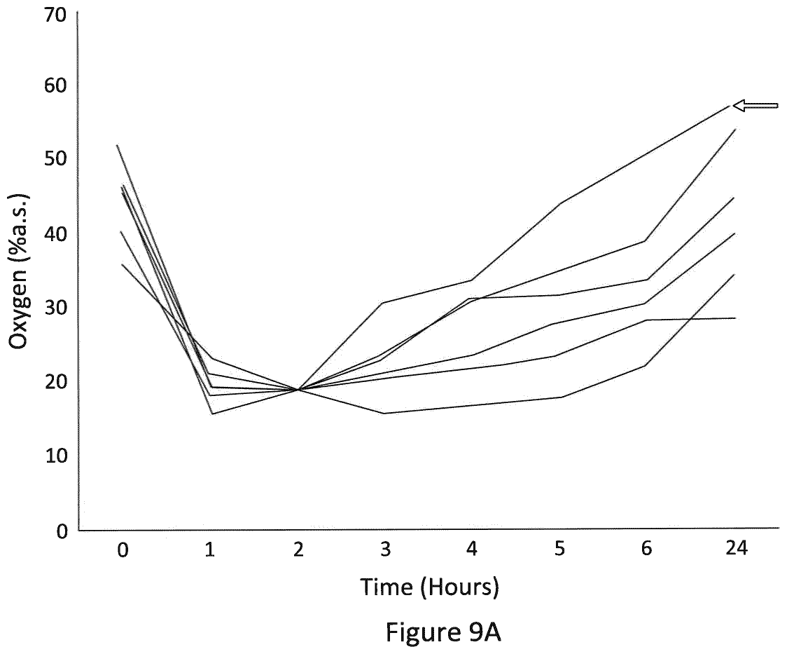

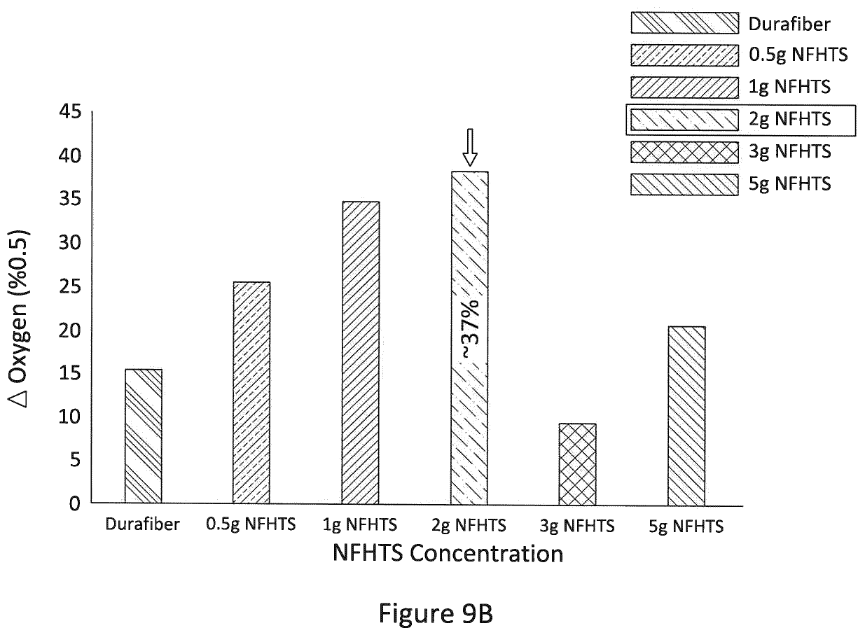

FIGS. 8-9 show the relative percent uptake and release of oxygen from multiple different perfluorocarbons over time as well as DURAFIBER and a control no dressing sample. FIGS. 8A and 8B show two model sets testing the relative percent oxygen uptake and release over time for multiple different perfluorocarbons.

In FIGS. 8A and 8B, pentafluorophenyltriethoxysilane, nonafluorohexyltriethoxysilane, 1H, 1H, 2H, 2H-perfluorodecyltriethoxysilane, and 1H, 1H, 2H, 2H-perfluorooctyltriethoxysilane were the perfluorocarbons tested, each separately bound to DURAFIBER, with DURAFIBER by itself used as a control. For the experiments in FIGS. 8A and 8B, the perfluorocarbon samples were enclosed in glass chambers where the oxygen concentration within the chamber was measured to determine the total oxygen uptake by the perfluorocarbon infused hydrogel samples. Over the 6 hour period, the nonafluorohexyltriethoxysilane sample absorbed the most oxygen from the chamber, and the overall oxygen concentration within the nonafluorohexyltriethoxysilane sample increased the most. In comparison to the DURAFIBER control sample, the nonafluorohexyltriethoxysilane sample absorbed roughly double the oxygen as the control.

For the experiments in 8C-8E, the nonafluorohexyltriethoxysilane bound to DURAFIBER samples were enclosed in glass chambers in parallel, with DURAFIBER by itself used as a control. For the experiments in FIG. 8C-E, the perfluorocarbon samples were enclosed in glass chambers where the oxygen concentration within the chamber was measured to determine the total oxygen uptake by the perfluorocarbon samples. Over the 6 hour period, the nonafluorohexyltriethoxysilane sample absorbed the most oxygen from the chamber, and the overall oxygen concentration within the nonafluorohexyltriethoxysilane sample increased the most. In comparison to the DURAFIBER control sample, the nonafluorohexyltriethoxysilane sample absorbed an increased amount of oxygen in comparison to the control.

FIGS. 9A and 9B illustrate the relative percent oxygen release for multiple different concentrations of nonafluorohexyltriethoxysilane bound to DURAFIBER, with DURAFIBER alone being the control sample. In this experiment, 0.5 g, 1.0 g, 2.0 g, 3.0 g, and 5.0 g of nonafluorohexyltriethoxysilane were each dissolved in separate 50 mL solutions containing an 85:15 ethanol: water ratio. 10 cm by 10 cm portions of DURAFIBER were added into the solutions and infused with nonafluorohexyltriethoxysilane. Once the nonafluorohexyltriethoxysilane was incorporated into DURAFIBER, with a DURAFIBER sample control, the infused samples were then tested to determine the relative oxygen release over time. The hydrogel samples infused in the 1.0 g and 2.0 g nonafluorohexyltriethoxysilane solutions had the best oxygen release, with the 2.0 g nonafluorohexyltriethoxysilane solution sample slightly outperforming the 1.0 g nonafluorohexyltriethoxysilane solution sample. In certain examples, a 10 cm×10 cm DURAFIBER may be loaded with varying amounts of perfluorocarbon such as about: 0.1 to 2.0 g, 0.2 to 1.8 g, 0.4 to 1.6 g, 0.6 to 1.4 g, 0.8 to 1.2 g, or about 1 g. In certain examples, a 10 cm×10 cm DURAFIBER may be loaded with 0.9-1.4 g perfluorocarbon, such as about 1.2 g.

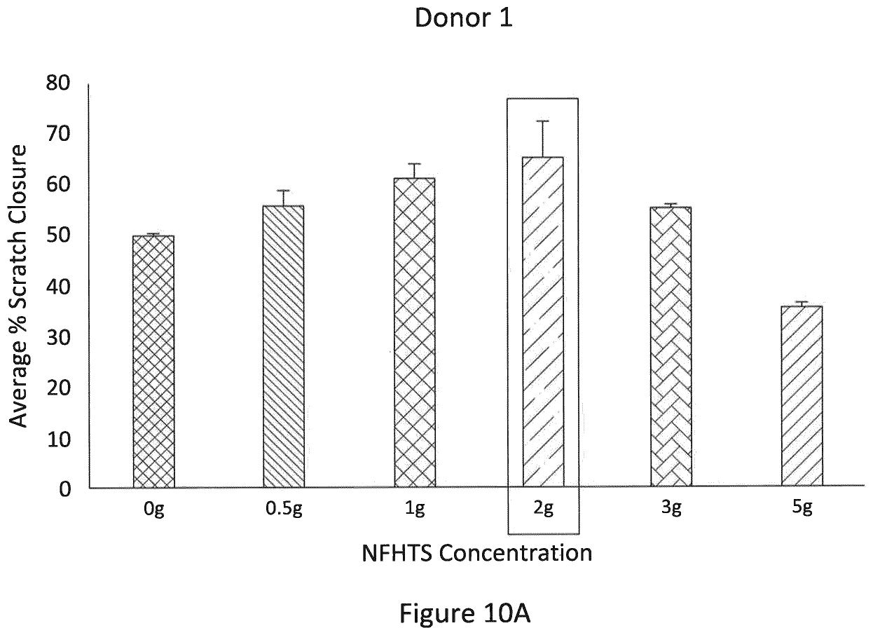

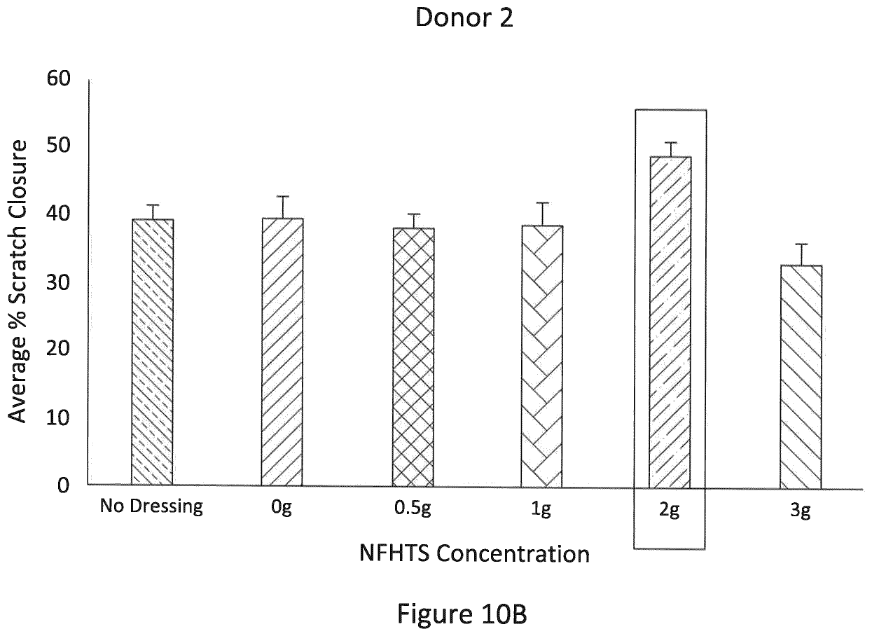

FIGS. 10A and 10B illustrates the relative percent wound closure across different donors as well as intra-donor for different concentrations of nonafluorohexyltriethoxysilane infused into samples for two separate donors. In this experiment, 0.5 g, 1.0 g, 2.0 g, 3.0 g, and 5.0 g of nonafluorohexyltriethoxysilane were each dissolved in separate 50 mL solutions containing an 85:15 ethanol:water ratio. 10 cm by 10 cm portions of DURAFIBER with hydrogel were added into the solutions and infused with nonafluorohexyltriethoxysilane. Once the nonafluorohexyltriethoxysilane was infused into separate DURAFIBER samples, with a DURAFIBER sample control, the infused samples were then tested to determine the relative cell migration and wound closure for each sample due to oxygen release. Mirroring the results from FIGS. 9A and 9B, the hydrogel sample infused in the 2.0 g nonafluorohexyltriethoxysilane solution showed the greatest cell migration and wound closure of the samples, followed by the hydrogel sample infused in the 1.0 g nonafluorohexyltriethoxysilane solution hydrogel sample.

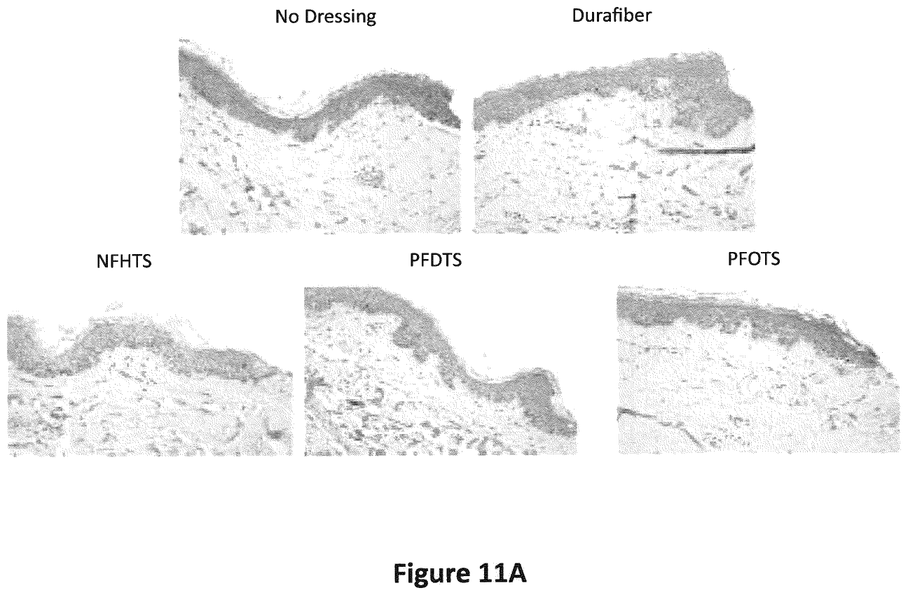

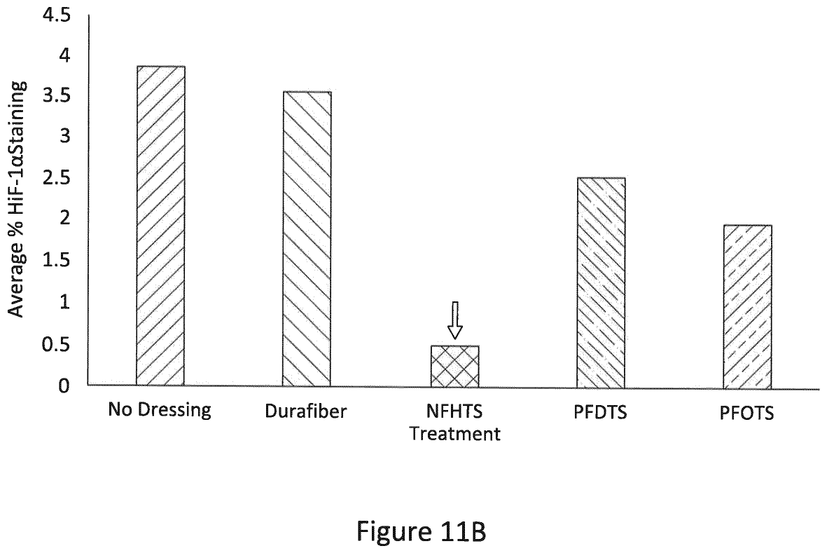

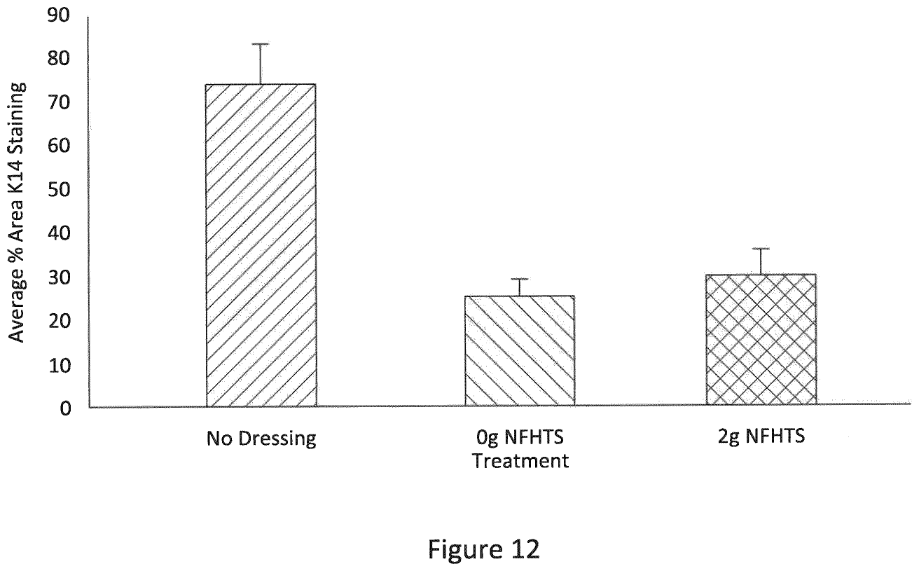

FIGS. 11A, 11B, and 12 illustrate an ex vivo study completed on perfluorocarbon wound dressings, determining epidermal proliferation and re-epithelialization over 24 and 48 hour time periods for different perfluorocarbons and varying concentrations of nonafluorohexyltriethoxysilane. For the experiment in FIGS. 11A and 11B, samples DURAFIBER were bound with nonafluorohexyltriethoxysilane, 1H, 1H, 2H, 2H-perfluorodecyltriethoxysilane, and 1H, 1H, 2H, 2H-perfluorooctyltriethoxysilane, with both a no dressing and DURAFIBER dressing alone control. Each sample was loaded a single time with oxygen and placed on a wound site. After a period of 24 hours, the wound area was biopsied and stained with HIF-1α, a marker that can indicate tissue hypoxia. The lowest concentration of HIF-1α found in any sample was the nonafluorohexyltriethoxysilane sample, at least three times lower than the next best perfluorocarbon sample. Since a low concentration of HIF-1α indicates an oxygen-rich tissue environment, the nonafluorohexyltriethoxysilane sample was the most effective at delivering oxygen directly to the wound site. The tissue samples shown in FIG. 11A display the increased regeneration with the nonafluorohexyltriethoxysilane sample, as only minimal staining was present. The minimal presence of HIF-1α, indicating an oxygen-rich tissue environment, was also seen in the graph in FIG. 11B.

FIG. 12 illustrates a similar ex vivo study as FIGS. 11A and 11B, only this experiment focused solely on nonafluorohexyltriethoxysilane infused hydrogel samples at varying concentrations. For the experiment in FIG. 12, a sample of DURAFIBER alone, a sample of 2.0 g nonafluorohexyltriethoxysilane bound to DURAFIBER, and a no dressing sample were prepared. Each sample was loaded with oxygen and placed on a wound site. After a period of 48 hours, wherein the samples were re-loaded with oxygen every 24 hours, the wound area was biopsied and underwent K14 wholemount staining, a marker that can indicate epidermal proliferation and re-epithelialization. The 2.0 g nonafluorohexyltriethoxysilane sample showed a positive effect on wound closure over the 48 hour period.

### Multilayered Wound Dressing with an Optional Integrated Source of Negative Pressure

In some embodiments, a source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. Additionally, some embodiments related to wound treatment comprising a wound dressing described herein may also be used in combination or in addition to those described in International Application WO 2016/174048 and International Patent Application PCT/EP2017/055225, filed on Mar. 6, 2017, entitled “WOUND TREATMENT APPARATUSES AND METHODS WITH NEGATIVE PRESSURE SOURCE INTEGRATED INTO THE WOUND DRESSING,” the disclosure of which is hereby incorporated by reference in its entirety herein, including further details relating to embodiments of wound dressings, the wound dressing components and principles, and the materials used for the wound dressings and wound dressing components.

The one of more perfluorocarbon layers may be constructed as any perfluorocarbon layer disclosed herein. The one or more perfluorocarbon layers may be constructed to be flexible but stiff enough to withstand negative pressure, such that the one or more perfluorocarbon layers are not collapsed excessively and thereby transmits negative pressure sufficiently to the wound when negative pressure is supplied to the wound dressing 100. The one or more perfluorocarbon layers may be constructed to include sufficient number or size of pores to enable transmission of negative pressure through it. Further, the one or more perfluorocarbon layers may have suitable thickness to transmit enough negative pressure to the wound. For example, the one or more perfluorocarbon layers may have a thickness of 1 mm to 10 mm, 1 mm to 7 mm, 1.5 to 7 mm, 1.5 mm to 4 mm, or 2 mm to 3 mm. In some embodiments, the one or more perfluorocarbon layers may have a thickness of approximately 2 mm.

A cover layer or backing layer 105 can be positioned over the transmission layer 604. The backing layer 105 can form a seal to the wound contact layer 104 at a perimeter region enclosing the transmission layer 604, the absorbent layer 401, and membrane electrolytic assembly 101. In some embodiments, the backing layer 105 can be a flexible sheet of material that forms and molds around the dressing components when they are applied to the wound. In other embodiments, the backing layer 105 can be a material that is preformed or premolded to fit around the dressing components as shown in FIG. 6.

The absorbent layer 401 has a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

### Multi-Layered Wound Dressings for NPWT with a Wrapped Around Transmission Layer

FIGS. 2-6 illustrate embodiments of a TNP wound treatment device comprising a wound dressing. As stated above, the wound dressing 100 can be any wound dressing embodiment disclosed herein or have any combination of features of any number of wound dressing embodiments disclosed herein. For example, the wound dressing 100 may be similar to a PICO single unit dressing available from Smith & Nephew as described previously. Embodiments of the wound dressings, wound dressing components, wound treatment apparatuses and methods described herein may also be used in combination or in addition to those described in International Publication No. WO 2017/114745 A1, published Jul. 6, 2017, titled “NEGATIVE PRESSURE WOUND THERAPY APPARATUS,” the disclosure of which is hereby incorporated by reference in its entirety.

The cover layer 105, which can be more clearly seen in FIG. 6, can be formed of substantially fluid impermeable material, such as film. The cover layer 105 can be similar to the cover layer or backing layer described in FIGS. 4-6 previously. The film may be transparent, such that other layers underneath the cover layer are also visible. The cover layer can include an adhesive for securing the dressing to the surrounding skin or a wound contact layer. The dressing can utilize a wound contact layer 104 and an absorbent layer 401 within the dressing. The wound contact layer and the absorbent layer can be similar to the wound contact layer and absorbent layers described in FIGS. 2-6 previously. The wound contact layer can be configured to be in contact with the wound. The wound contact layer can include an adhesive on the patient facing side for securing the dressing to the surround skin or on the top side for securing the wound contact layer 104 to a cover layer 105 or other layer of the dressing. In operation, in some embodiments the wound contact layer can be configured to provide unidirectional flow so as to facilitate removal of exudate from the wound while blocking or substantially preventing exudate from returning to the wound. Further, an absorbent layer 401 for absorbing and retaining exudate aspirated from the wound can be utilized. In some embodiments, the absorbent layer can include an absorbent material, for example, a superabsorbent material or other absorbent material known in the art. In some embodiments, the absorbent layer can include a shaped form of a superabsorber layer with recesses or compartments for the pump, electronics, and accompanying components. In some embodiments, the wound dressing can include multiple absorbent layers.

The absorbent material 401 as shown in FIG. 4 which may be a foam or non-woven natural or synthetic material and which may optionally include or be super-absorbent material, forms a reservoir for fluid, particularly liquid, removed from the wound site and draws those fluids towards a cover layer 105. The material of the absorbent layer can be similar to the absorbent material described with reference to FIGS. 2-5. The material of the absorbent layer also prevents liquid collected in the wound dressing from flowing in a sloshing manner. The absorbent layer 401 also helps distribute fluid throughout the layer via a wicking action so that fluid is drawn from the wound site and stored throughout the absorbent layer. This helps prevent agglomeration in areas of the absorbent layer.

In some embodiments, the absorbent layer 401 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. Also, all regions of the absorbent layer are provided with liquid.

The wicking action also assists in bringing liquid into contact with the upper cover layer to aid increase transpiration rates of the dressing.

The wicking action also assists in delivering liquid downwards towards the wound bed when exudation slows or halts. This delivery process helps maintain the transmission layer or lower spacer layer and lower wound bed region in a moist state which helps prevent crusting within the dressing (which could lead to blockage) and helps maintain an environment optimized for wound healing.

In some embodiments, the absorbent layer 401 may be an air-laid material. Heat fusible fibers may optionally be used to assist in holding the structure of the pad together. It will be appreciated that rather than using super-absorbing particles or in addition to such use, super-absorbing fibers may be utilized according to certain embodiments of the present invention. An example of a suitable material is the Product Chem-Posite™ 11 C available from Emerging Technologies Inc (ETi) in the USA.

Optionally, according to certain embodiments of the present invention, the absorbent layer 401 may include synthetic stable fibers and/or bi-component stable fibers and/or natural stable fibers and/or super-absorbent fibers. Fibers in the absorbent layer may be secured together by latex bonding or thermal bonding or hydrogen bonding or a combination of any bonding technique or other securing mechanism. In some embodiments, the absorbent layer is formed by fibers which operate to lock super-absorbent particles within the absorbent layer. This helps ensure that super-absorbent particles do not move external to the absorbent layer and towards an underlying wound bed. This is particularly helpful because when negative pressure is applied there is a tendency for the absorbent pad to collapse downwards and this action would push super-absorbent particle matter into a direction towards the wound bed if they were not locked away by the fibrous structure of the absorbent layer.

The absorbent layer 401 may comprise a layer of multiple fibers. Preferably, the fibers are strand-like and made from cellulose, polyester, viscose or the like. Preferably, dry absorbent particles are distributed throughout the absorbent layer ready for use. In some embodiments, the absorbent layer comprises a pad of cellulose fibers and a plurality of super absorbent particles. In additional embodiments, the absorbent layer is a non-woven layer of randomly orientated cellulose fibers.

Super-absorber particles/fibers may be, for example, sodium polyacrylate or carbomethoxycellulose materials or the like or any material capable of absorbing many times its own weight in liquid. In some embodiments, the material can absorb more than five times its own weight of 0.9% W/W saline, etc. In some embodiments, the material can absorb more than 15 times its own weight of 0.9% W/W saline, etc. In some embodiments, the material is capable of absorbing more than 20 times its own weight of 0.9% W/W saline, etc. Preferably, the material is capable of absorbing more than 30 times its own weight of 0.9% W/W saline, etc.

Preferably, the particles of superabsorber are very hydrophilic and grab the fluid as it enters the dressing, swelling up on contact. An equilibrium is set up within the dressing core whereby moisture passes from the superabsorber into the dryer surrounding area and as it hits the top film the film switches and the fluid vapor starts to be transpired. A moisture gradient is established within the dressing to continually remove fluid from the wound bed and ensure the dressing does not become heavy with exudate.

The absorbent layer 401 can include at least one through hole. Although not essential to certain embodiments of the present invention the use of through holes in the super-absorbent layer provide a fluid flow pathway which is particularly unhindered and this is useful in certain circumstances.

Use of one or more through holes in the absorption layer 401 also has the advantage that during use if the absorbent layer contains a gel forming material, such as superabsorber, that material as it expands to absorb liquid, does not form a barrier through which further liquid movement and fluid movement in general cannot pass. In this way each opening in the absorbent layer provides a fluid pathway between the lower transmission or spacer layer and the upper transmission or spacer layer to the wound facing surface of the filter.

These layers can be covered with one layer of a film or cover layer 105. The cover layer can include a filter that can be positioned over the absorbent layer as described in International Application Publication No. WO 2013/175306 A2, U.S. Publication No. US2011/0282309, and U.S. Publication No. 2016/0339158 the entirety of which is hereby incorporated by reference. As shown in FIGS. 4-6 gas impermeable, but moisture vapor permeable, cover layer 105 extends across the width of the wound dressing. The cover layer may be similar to the cover layer or backing layer described with reference to FIGS. 2-6. The cover layer 105, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way an effective chamber is made between the cover layer and a wound site where a negative pressure can be established. The cover layer 105 is sealed to the wound contact layer 104 in a border region around the circumference of the dressing, ensuring that no air is drawn in through the border area, for example via adhesive or welding techniques. The cover layer 105 protects the wound from external bacterial contamination (bacterial barrier) and allows liquid from wound exudates to be transferred through the layer and evaporated from the film outer surface. The cover layer 105 typically comprises two layers: a polyurethane film and an adhesive pattern spread onto the film. The polyurethane film is moisture vapor permeable and may be manufactured from a material that has an increased water transmission rate when wet.

Negative pressure can be lost at the wound bed when free absorbent capacity remains in the dressing. This can occur because some or all of the pores in the filter are blocked with liquid or particulates. In some embodiments, solutions are utilized to allow the full capacity of the dressing absorbent layer to be utilized whilst maintaining the air path between the source of negative pressure and the wound bed.

In dressing embodiments that utilize a cover layer directly over the absorbent layer the dressing can have a void underneath the filter which can fill with liquid, thus blocking the filter pores and preventing air flow to the wound bed. A spacer layer or transmission layer 604 can be used to provide a fluid flow path above the absorbent layer 401. In some embodiments, the transmission layer 604 in the dressing can be provided above and below the absorbent layer. The transmission layer can be incompressible and maintain a path for fluid flow between the source of negative pressure and the wound bed, via the filter. In some embodiments, the transmission layer can encapsulate or wrap around the absorbent layer as shown in FIGS. 2-6. The transmission layer can traverse the length of the top surface of the absorbent layer and wrap around at least one side of the absorbent layer and traverse the length of the bottom surface (wound facing surface) of the absorbent layer. In some embodiments, the transmission layer can wrap around two sides of the absorbent layer as shown in FIGS. 2-6.

In some embodiments, the transmission layer can be utilized to assist in distributing negative pressure over the wound site and facilitating transport of wound exudate and fluids into the wound dressing.

A lower portion of the transmission layer 604 of porous material can be located above the wound contact layer and below the absorbent layer and wrapped around the edges of the absorbent layer. As the transmission layer is wrapped around at least one edge of the absorbent layer, the transmission layer has an upper portion of the transmission layer that can be positioned between the cover layer and the absorbent layer. As used herein the edge of the absorbent layer or the dressing refers to the sides of the material that are substantially perpendicular to the wound surface and run along the height of the material.

In some embodiments, the transmission layer can be a porous layer. This spacer layer, or transmission layer 604 allows transmission of fluid including liquid and gas away from a wound site into upper layers of the wound dressing as described with reference to FIG. 6. In particular, the transmission layer 604 ensures that an open-air channel can be maintained to communicate negative pressure over the wound area even when the absorbent layer has absorbed substantial amounts of exudates. The layer should remain open under the typical pressures that will be applied during negative pressure wound therapy as described previously, so that the whole wound site sees an equalized negative pressure. The transmission layer 604 may be formed of a material having a three-dimensional structure. For example, a knitted or woven spacer fabric (for example Baltex 7970 weft knitted polyester) or a non-woven fabric could be used. Other materials, such as those described previously herein, could of course be utilized.

The wound dressing 100 may incorporate or comprise one or more perfluorocarbon layers as described herein. One of skill in the art will understand that the wound dressing 100 may incorporate any of the one or more perfluorocarbon layers disclosed herein this section or elsewhere in the specification. One of skill in the art will also understand that the one or more perfluorocarbon layers may be incorporated as a whole component layer or a part of a component layer. In some embodiments, the one or more perfluorocarbon layers may be provided below the transmission layer 604. In some embodiments, the one or more perfluorocarbon layers may be provided above the wound contact layer 104. In some embodiments, the one or more perfluorocarbon layers may replace all or part of the transmission layer 604, for example such that the one or more perfluorocarbon layers wraps around the edges of the absorbent layer 401 (described further below) and the wound contact layer 104. In some embodiments, the one or more perfluorocarbon layers can supplement or replace the absorbent layer 401.

As shown in FIGS. 2-6, a wound dressing 100 can include a wound contact layer 104. In some embodiments, the wound contact layer 104 can be a double-face coated (silicone-acrylic) perforated adhesive wound contact layer. A transmission layer 604 and absorbent layer 401 can be provided similar to the dressing described with reference to FIGS. 2-6 but the transmission layer 604 over-borders the absorbent layer 401. The transmission layer 604 can over-border the absorbent layer by 5 mm at the perimeter. This can be the reverse of the cut geometry in the dressings as described previously. In some embodiments, there is no through-hole or aperture in the absorbent layer 401 or transmission layer 604. In some embodiments, the hole in the absorbent layer could be disadvantageous because it could become filled with superabsorbent particles or other material and block the filter in the standard dressing. In some embodiments, the transmission layer 604 can include a 3D fabric.

The wound dressing 100 may incorporate or comprise one or more perfluorocarbon layers as described herein this section or elsewhere in the specification. One of skill in the art will understand that the wound dressing 300 may incorporate any of the one or more perfluorocarbon layers disclosed herein this section or elsewhere in the specification. One of skill in the art will also understand that the one or more perfluorocarbon layers may be incorporated as a whole component layer or a part of a component layer. In some embodiments, the one or more perfluorocarbon layers may be provided below the transmission layer 604. In some embodiments, the one or more perfluorocarbon layers may be provided above the wound contact layer 104. In some embodiments, the one or more perfluorocarbon layers may replace the transmission layer 604. In some embodiments, the one or more nitric oxide generating layers can supplement or replace the absorbent layer 401.

The one or more perfluorocarbon layers may be constructed such as disclosed elsewhere herein, for example to incorporate nonafluorohexyltriethoxysilane, pentafluorophenyltriethoxysilane, 1H, 1H, 2H, 2H-perfluorodecyltriethoxysilane, and/or 1H, 1H, 2H, 2H-perfluorooctyltriethoxysilane. The one or more perfluorocarbon layers may be constructed to be flexible but stiff enough to withstand negative pressure, such that the one or more perfluorocarbon infused hydrogel is not collapsed excessively and thereby transmits negative pressure sufficiently to the wound when negative pressure is supplied to the wound dressing 100.

The one or more perfluorocarbon layers may be constructed to include sufficient number or size of pores to enable transmission of negative pressure through it. Further, the one or more perfluorocarbon layers may have suitable thickness to transmit enough negative pressure to the wound. For example, the one or more perfluorocarbon layers may have a thickness of 1 mm to 10mm, 1 mm to 7 mm, 1.5 mm to 7 mm, 1.5 mm to 4 mm, or 2 mm to 3 mm. In some embodiments, the one or more perfluorocarbon layers may have a thickness of approximately 2 mm.

In some embodiments, the one or more perfluorocarbon layers 102 may be infused with nonafluorohexyltriethoxysilane. In some embodiments, the one or more perfluorocarbon layers 102 may be infused with pentafluorophenyltriethoxysilane. In some embodiments, the one or more perfluorocarbon layers 102 may be infused with 1H, 1H, 2H, 2H-perfluorodecyltriethoxysilane. In some embodiments, the one or more perfluorocarbon layers 102 may be infused with 1H, 1H, 2H, 2H-perfluorooctyltriethoxysilane.

The absorbent layer 401 has a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

### Multi-Layered Wound Dressings for NPWT with an Obscuring Layer

FIG. 6 illustrates a cross-section through a wound dressing 100 similar to the wound dressing of FIGS. 2-6 according to an embodiment of the disclosure. The wound dressing 100, which can alternatively be any wound dressing embodiment disclosed herein including without limitation wound dressing 100 or any combination of features of any number of wound dressing embodiments disclosed herein, can be located over a wound site to be treated. The dressing 100 may be placed to as to form a sealed cavity over the wound site. In a preferred embodiment, the dressing 100 comprises a backing layer 105 attached to a wound contact layer 104, similar to the cover layer and wound contact layer described with reference to FIGS. 2-6. These two layers 105, 104 are preferably joined or sealed together so as to define an interior space or chamber. This interior space or chamber may comprise additional structures that may be adapted to distribute or transmit negative pressure, store wound exudate and other fluids removed from the wound, and other functions as described herein. Examples of such structures, described below, include a transmission layer 604 and an absorbent layer 401, similar to the transmission layer and absorbent layer described with reference to FIGS. 4-6.

A layer of porous material can be located above the wound contact layer 104. This porous layer, or transmission layer, 604 allows transmission of fluid including liquid and gas away from a wound site into upper layers of the wound dressing. In particular, the transmission layer 604 preferably ensures that an open-air channel can be maintained to communicate negative pressure over the wound area even when the absorbent layer has absorbed substantial amounts of exudates. The layer 604 should preferably remain open under the typical pressures that will be applied during negative pressure wound therapy as described above, so that the whole wound site sees an equalized negative pressure.

In some embodiments, the layer 604 may be formed of a material having a three-dimensional structure. For example, a knitted or woven spacer fabric (for example Baltex 7970 weft knitted polyester) or a non-woven fabric could be used.

A layer of absorbent material 401 is provided above the transmission layer 604. The absorbent material, which comprise a foam or non-woven natural or synthetic material, and which may optionally comprise a super-absorbent material, forms a reservoir for fluid, particularly liquid, removed from the wound site. In some embodiments, the layer 401 may also aid in drawing fluids towards the backing layer 105.

With reference to FIG. 5, a masking or obscuring layer 501 can be positioned beneath at least a portion of the backing layer 105. In some embodiments, the obscuring layer 501 can have any of the same features, materials, or other details of any of the other embodiments of the obscuring layers disclosed herein, including but not limited to having any viewing windows or holes. Examples of wound dressings with obscuring layers and viewing windows are described in International Patent Publication WO2014/020440, the entirety of which is incorporated by reference in its entirety. Additionally, the obscuring layer 501 can be positioned adjacent to the backing layer, or can be positioned adjacent to any other dressing layer desired. In some embodiments, the obscuring layer 501 can be adhered to or integrally formed with the backing layer. Preferably, the obscuring layer 501 is configured to have approximately the same size and shape as the absorbent layer 401 so as to overlay it. As such, in these embodiments the obscuring layer 2107 will be of a smaller area than the backing layer 105.

The material of the absorbent layer 401 may also prevent liquid collected in the wound dressing 100 from flowing freely within the dressing, and preferably acts so as to contain any liquid collected within the absorbent layer 401. The absorbent layer 401 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 401 may typically be manufactured from ALLEVYN™ foam, Freudenberg 114-224-4 and/or Chem-Posite™ 11C-450. In some embodiments, the absorbent layer 401 may comprise a composite comprising superabsorbent powder, fibrous material such as cellulose, and bonding fibers. In a preferred embodiment, the composite is an air-laid, thermally-bonded composite.

The backing layer 105 is preferably gas impermeable, but moisture vapor permeable, and can extend across the width of the wound dressing 100. The backing layer 105, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way an effective chamber is made between the backing layer 105 and a wound site where a negative pressure can be established. The backing layer 105 is preferably sealed to the wound contact layer 104 in a border region around the circumference of the dressing, ensuring that no air is drawn in through the border area, for example via adhesive or welding techniques. The backing layer 105 protects the wound from external bacterial contamination (bacterial barrier) and allows liquid from wound exudates to be transferred through the layer and evaporated from the film outer surface. The backing layer 105 preferably comprises two layers; a polyurethane film and an adhesive pattern spread onto the film. The polyurethane film is preferably moisture vapor permeable and may be manufactured from a material that has an increased water transmission rate when wet.

In some embodiments, the absorbent layer 401 may be of a greater area than the transmission layer 604, such that the absorbent layer overlaps the edges of the transmission layer 604, thereby ensuring that the transmission layer does not contact the backing layer 105. This provides an outer channel of the absorbent layer 401 that is in direct contact with the wound contact layer 2102, which aids more rapid absorption of exudates to the absorbent layer. Furthermore, this outer channel ensures that no liquid is able to pool around the circumference of the wound cavity, which may otherwise seep through the seal around the perimeter of the dressing leading to the formation of leaks.

The wound dressings 100 may incorporate or comprise one or more perfluorocarbon layers as described herein this section or elsewhere in the specification. One of skill in the art will understand that the wound dressing 100 may incorporate any of the one or more perfluorocarbon layers disclosed herein this section or elsewhere in the specification. In some embodiments, the one or more perfluorocarbon layers may be provided below the transmission layer 604. In some embodiments, the one or more perfluorocarbon infused hydrogel may be provided above the wound contact layer 104. In some embodiments, the one or more perfluorocarbon layers may replace the transmission layer 604, such that the one or more perfluorocarbon layers is provided between an absorbent layer 401 (described further below) and the wound contact layer 104. In some embodiments, the one or more perfluorocarbon layers may be the lowermost layer of the wound dressing 100. The one or more perfluorocarbon layers may have same or substantially similar size and shape with the transmission layer 604 and/or the absorbent layer 401. In some embodiments, the one or more perfluorocarbon layers 102 can supplement or replace the absorbent layer 401.

The one or more nitric oxide generating layers may be constructed to be flexible but stiff enough to withstand negative pressure, such that the one or more nitric oxide generating layers are not collapsed excessively and thereby transmits negative pressure sufficiently to the wound when negative pressure is supplied to the wound dressing 100. The one or more nitric oxide generating layers may be constructed to include sufficient number or size of pores to enable transmission of negative pressure through it. Further, the one or more nitric oxide generating layers may have a suitable thickness to transmit enough negative pressure to the wound. For example, the one or more nitric oxide generating layers may have a thickness of 1 mm to 10 mm, 1 mm to 7 mm, 1.5 mm to 7 mm, 1.5 mm to 4 mm, or 2 mm to 3 mm. In some embodiments, the one or more nitric oxide generating layers may have a thickness of approximately 2 mm.

The wound dressing 100 can be constructed similar to the embodiments of FIG. 6 above, and may comprise an absorbent material 401 underneath or within a backing layer 105. Optionally, a wound contact layer and a transmission layer may also be provided as part of the wound dressing 100 as described above with reference to FIG. 6A. The absorbent material 401 can contain a narrowed central or waisted portion to increase flexibility and conformability of the wound dressing to the skin surface. The backing layer 105 may have a border region that extends beyond the periphery of the absorbent material 401. The backing layer 105 may be a translucent or transparent backing layer, such that the border region created from the backing layer 105 can be translucent or transparent. The area of the border region of the backing layer 105 can be approximately equal around the perimeter of the entire dressing with the exception of the narrowed central portion, where the area of the border region is larger. One will recognize that the size of the border region will depend on the full dimensions of the dressing and any other design choices.

As illustrated in FIG. 6, provided at least at the top of or over the absorbent layer 401 and under the backing layer 105 may be an obscuring layer 501 that optionally has one or more viewing windows. The obscuring layer 501 may partially or completely obscure contents (such as fluids) contained within the wound dressing 100 and/or the absorbent material (i.e., within the absorbent material 401 or under the backing layer 105). The obscuring layer may be a colored portion of the absorbent material, or may be a separate layer that covers the absorbent material. In some embodiments, the absorbent material 401 may be hidden (partially or completely), colored, or tinted, via the obscuring layer 501, so as to provide cosmetic and/or aesthetic enhancements, in a similar manner to what is described above. The obscuring layer is preferably provided between the topmost backing layer 105 and the absorbent material 401, although other configurations are possible. Other layers and other wound dressing components can be incorporated into the dressing as herein described.

The obscuring layer 501 can be positioned at least partially over the absorbent material 401. In some embodiments, the obscuring layer 501 can be positioned adjacent to the backing layer, or can be positioned adjacent to any other dressing layer desired. In some embodiments, the obscuring layer 501 can be adhered to or integrally formed with the backing layer and/or the absorbent material.

As illustrated in FIGS. 5 and 6, the obscuring layer 501 can have substantially the same perimeter shape and size as the absorbent material 401. The obscuring layer 501 and absorbent material 401 can be of equal size so that the entirety of the absorbent material 401 can be obscured by the obscuring layer 501. The obscuring layer 501 may allow for obscuring of wound exudate, blood, or other matter released from a wound. Further, the obscuring layer 501 can be completely or partially opaque having cut-out viewing windows or perforations.

In some embodiments, the obscuring layer 501 can help to reduce the unsightly appearance of a dressing during use, by using materials that impart partial obscuring or masking of the dressing surface. The obscuring layer 501 in one embodiment only partially obscures the dressing, to allow clinicians to access the information they require by observing the spread of exudate across the dressing surface. The partial masking nature of this embodiment of the obscuring layer enables a skilled clinician to perceive a different color caused by exudate, blood, by-products etc. in the dressing allowing for a visual assessment and monitoring of the extent of spread across the dressing. However, since the change in color of the dressing from its clean state to a state containing exudate is only a slight change, the patient is unlikely to notice any aesthetic difference. Reducing or eliminating a visual indicator of wound exudate from a patient's wound is likely to have a positive effect on their health, reducing stress for example.

In some embodiments, the obscuring layer can be formed from a non-woven fabric (for example, polypropylene), and may be thermally bonded using a diamond pattern with 19% bond area. In various embodiments, the obscuring layer can be hydrophobic or hydrophilic. Depending on the application, in some embodiments, a hydrophilic obscuring layer may provide added moisture vapor permeability. In some embodiments, however, hydrophobic obscuring layers may still provide sufficient moisture vapor permeability (i.e., through appropriate material selection, thickness of the obscuring layer), while also permitting better retention of dye or color in the obscuring layer. As such, dye or color may be trapped beneath the obscuring layer. In some embodiments, this may permit the obscuring layer to be colored in lighter colors or in white. In the preferred embodiment, the obscuring layer is hydrophobic. In some embodiments, the obscuring layer material can be sterilizable using ethylene oxide. Other embodiments may be sterilized using gamma irradiation, an electron beam, steam or other alternative sterilization methods. Additionally, in various embodiments the obscuring layer can colored or pigmented, e.g., in medical blue. The obscuring layer may also be constructed from multiple layers, including a colored layer laminated or fused to a stronger uncolored layer. Preferably, the obscuring layer is odorless and exhibits minimal shedding of fibers.

The absorbent layer 401, itself may be colored or tinted in some embodiments, however, so that an obscuring layer is not necessary. The dressing may optionally include a means of partially obscuring the top surface. This could also be achieved using a textile (knitted, woven, or non-woven) layer without openings, provided it still enables fluid evaporation from the absorbent structure. It could also be achieved by printing an obscuring pattern on the top film, or on the top surface of the uppermost pad component, using an appropriate ink or colored pad component (yarn, thread, coating) respectively. Another way of achieving this would be to have a completely opaque top surface, which could be temporarily opened by the clinician for inspection of the dressing state (for example through a window), and closed again without compromising the environment of the wound. One or more viewing windows preferably extend through the obscuring layer 501. These viewing windows may allow visualization by a clinician or patient of the wound exudate in the absorbent material below the obscuring layer. In a preferred embodiment, two or more viewing windows may be parallel with one or more sides of the dressing 100. In some embodiments, the one or more viewing windows may measure between 0.1 mm and 20 mm, preferably 0.4 mm to 10 mm, and even more preferably, 1 mm to 4 mm. The viewing windows may be cut through the obscuring layer 501 or may be part of an uncolored area of the obscuring layer 501 and therefore may allow visualization of the absorbent material 401. The one or more viewing windows can be arranged in a repeating pattern across the obscuring layer 501 or can be arranged at random across the obscuring layer. Additionally, the one or more viewing windows can be a circular shape or dots. In some embodiments, the viewing windows correspond to the area of the absorbent material 401 that is not covered by the obscuring layer 501. As such, the absorbent material 401 is directly adjacent the backing layer 105 in this area. Since the obscuring layer 501 acts as a partial obscuring layer, the viewing windows may be used by a clinician or other trained user to assess the spread of wound exudate throughout the dressing. In some embodiments, the viewing windows can comprise an array of dots or crescent shaped cut-outs. Additionally, in some embodiments, the dot pattern can be distributed evenly throughout the obscuring layer and across the entire or substantially the entire surface of the obscuring layer. In some embodiments, the viewing windows may be distributed randomly throughout the obscuring layer. Preferably, the area of the obscuring layer 501 uncovered by the one or more viewing windows is balanced to as to minimize the appearance of exudate while permitting the inspection of the dressing 100 and/or absorbent material 401. In some embodiments, the area exposed by the one or more viewing windows does not exceed 20% of the area of the obscuring layer 501, preferably 10%, and even more preferably 5%.

The viewing windows may take several configurations. In some embodiments, the viewing windows may comprise an array of regularly spaced uncolored dots (holes) made into the obscuring layer 501. While the dots illustrated here are in a particular pattern, the dots may be arranged in different configurations, or at random. The viewing windows are preferably configured so as to permit a patient or caregiver to ascertain the status of the absorbent layer 401, in particular to determine its saturation level, as well as the color of the exudate (e.g., whether excessive blood is present). By having one or more viewing windows, the status of the absorbent layer 401 can be determined in an unobtrusive manner that is not aesthetically unpleasing to a patient. Because a large portion of the absorbent layer 401 may be obscured, the total amount of exudate may therefore be hidden. As such, the status and saturation level of the absorbent layer 401 may therefore present a more discreet external appearance so as to reduce patient embarrassment and visibility and thereby enhance patient comfort. In some configurations, the one or more viewing windows may be used to provide a numerical assessment of the degree of saturation of the dressing 100. This may be done electronically (e.g., via a digital photograph assessment), or manually. For example, the degree of saturation may be monitored by counting the number of viewing windows which may be obscured or tinted by exudate or other wound fluids.

In some embodiments, the absorbent layer 401 or the obscuring layer 501, in particular the colored portion of the absorbent layer, may comprise (or be colored because of) the presence of an auxiliary compound. The auxiliary compound may in some embodiments be activated charcoal, which can act to absorb odors. The use of antimicrobial, antifungal, anti-inflammatory, and other such therapeutic compounds is also possible. In some embodiments, the color may change as a function of time (e.g., to indicate when the dressing needs to be changed), if the dressing is saturated, or if the dressing has absorbed a certain amount of a harmful substance (e.g., to indicate the presence of infectious agents). In some embodiments, the one or more viewing windows may be monitored electronically, and may be used in conjunction with a computer program or system to alert a patient or physician to the saturation level of the dressing 100.

In some embodiments, the one or more perfluorocarbon layers 102 may incorporate nonafluorohexyltriethoxysilane. In some embodiments, the one or more perfluorocarbon layers 102 may incorporate pentafluorophenyltriethoxysilane. In some embodiments, the one or more perfluorocarbon layers 102 may incorporate 1H, 1H, 2H, 2H-perfluorodecyltriethoxysilane. In some embodiments, the one or more perfluorocarbon layers 102 may incorporate 1H, 1H, 2H, 2H-perfluorooctyltriethoxysilane.

The absorbent layer 401 has a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

The wound dressing 100 also includes a wound contact layer 104, which may include a perforated film. The perforated film is located adjacent to the wound contact layer and helps to prevent the wound dressing 100 from attaching to the wound as the wound heals. For example, the perforated film can help prevent fibres of the absorbent layer 401 from becoming embedded in the wound. Perforations in the perforated film are aptly substantially uniformly distributed and are of suitable size to allow passage of exudate into the wound dressing 100, e.g. with holes having a diameter of 1-2.5 mm. The perforated film is aptly formed from polyurethane. The wound contact layer 104 may also include an adhesive located under the perforated film (i.e. on the wound facing side of the perforated film) for adhering the wound dressing 100 to the skin. In this case the adhesive is silicone 3318 and is aptly spread onto the underside of the perforated film with a coat weight of around 30-200 g/m². In some other examples, an additional attachment element, for example bandages, strips of tape, or compression bandages may be used to secure the wound dressing 100 to the patient.

The top side of the perforated film (i.e. the side distal from the wound) may be coated with a further adhesive layer. Aptly, the further adhesive layer may be an acrylic adhesive, though other suitable adhesives may also be used. In other examples the wound contact layer 104 may be laminated (e.g. heat laminated) without the need for the further adhesive layer in between.

### Perfluorocarbon Layers

FIGS. 2-6 illustrate a wound dressing 100 including perfluorocarbon layers according to some embodiments. In the illustrated embodiments, the wound dressing 100 may include a cover layer 105, a wound contact layer 104, one or more perfluorocarbon layers 102, one or more dressing layers 102, an obscuring or masking layer 501, a transmission layer 604, an absorption layer 401, and a membrane electrolytic assembly 101. In some embodiments, the wound dressing 100 may include additional layers, as further described herein. One of skill in the art will understand that although the various sections of the dressing may be referred to as “layers,” such sections may be in other suitable shapes or configurations.

The cover layer 105 may be gas impermeable, but moisture vapor permeable, and can extend across the width of the wound dressing 100. The cover layer 105, which may for example be a polyurethane film (for example, Elastollan SP9109 or Elastollan SP806) having a pressure sensitive adhesive on one side, may be impermeable to gas and this layer may thus operate to cover the wound and to seal a wound cavity over which the wound dressing is placed. Therefore a chamber or a sealed wound space is made between the cover layer 105 and the wound site. In some embodiments, negative pressure can be established within the chamber or the sealed wound space made between the cover layer 105 and the wound site. The cover layer 105 protects the wound from external bacterial contamination (bacterial barrier) and allows liquid from wound exudates to be transferred through the layer and evaporated from the film outer surface. The cover layer 105 may include two or more layers, for example, a polyurethane film and an adhesive pattern spread onto the film. In certain examples, the polyurethane film may be moisture vapor permeable and may be manufactured from a material that has an increased water transmission rate when wet. In some embodiments, the moisture vapor permeability of the cover layer increases when the cover layer becomes wet. The moisture vapor permeability of the wet cover layer may be up to about ten times more than the moisture vapor permeability of the dry cover layer. In some embodiments, the cover layer 105 may be replaced or supplemented with an additional wound dressings described elsewhere herein, such that the additional wound dressings are positioned above the one or more perfluorocarbon layers.

In some embodiments, the one or more perfluorocarbon layers 102 may incorporate nonafluorohexyltriethoxysilane. In some embodiments, the one or more perfluorocarbon layers 102 may incorporate pentafluorophenyltriethoxysilane. In some embodiments, the one or more perfluorocarbon layers 102 may incorporate 1H, 1H, 2H, 2H-perfluorodecyltriethoxysilane. In some embodiments, the one or more perfluorocarbon layers 102 may incorporate 1H, 1H, 2H, 2H-perfluorooctyltriethoxysilane.

The absorbent layer 401 has a fibrous structure for absorbing exudate from a wound site. In this example, the absorbent layer 401 includes superabsorbent fibres. The absorbent layer 401 also includes other fibres. In this example, the absorbent layer includes superabsorbent fibres, viscose fibres and polyester fibres. In this example, the absorbent layer 401 includes around 40% superabsorbent fibres, 40% viscose fibres, and 20% polyester fibres. In other examples, the absorbent layer may include around 0-50% superabsorbent fibres, 0-100% viscose fibres and 0-50% polyester fibres. Suitable superabsorbent fibres include crosslinked acrylate copolymer fibres that are partially neutralized to sodium salt however other superabsorbent fibres are available. The absorbent layer 401 may be manufactured using a needling process in which the fibres are mechanically tangled together. In other examples, the absorbent layer 401 may include other ratios of superabsorbent, viscose and polyester fibres. For example, the absorbent layer may include around 50% superabsorbent fibres, 35% viscose fibres and 20% polyester fibres. Alternatively, the absorbent layer may include 40% superabsorbent fibres and 60% viscose fibres.

The one or more perfluorocarbon layers 102 may include a mesh, a foam, a gel or any other material suitable for containing the perfluorocarbon infused hydrogel. For example, the one or more perfluorocarbon layers 102 may include a mesh. The mesh may be knitted, woven or non-woven. The mesh may be made of a polymeric material, for example, viscose, polyamide, polyester, polypropylene or a combination thereof. In some embodiments, the one or more perfluorocarbon layers 102 may include polypropylene, polyester, polyurethane, polyvinyl chloride, polyamide, viscose, polyester, polypropylene and/or cellulose. As described herein, the one or more perfluorocarbon layers 102 may be constructed from one or more polymers. In some embodiments, the one or more perfluorocarbon layers 102 may be constructed from a colored material, such that the one or more perfluorocarbon layers 102 can be visible to assist positioning of the wound dressing 100 during application to the wound, and to reduce the risk of incomplete removal of the one or more perfluorocarbon layers 102 from the wound after treatment. The one or more perfluorocarbon layers 102 may be fully or semi-permeable to the diffusion of oxygen.

In some embodiments, the one or more perfluorocarbon layers 102 are the lowermost layers of the dressing 100, such that the one or more perfluorocarbon layers 102 may contact the wound. In some embodiments, the one or more perfluorocarbon layers 102 may be positioned within and/or over the wound. The one or more perfluorocarbon layers 102 may be constructed such that the one or more perfluorocarbon layers 102 do not substantially adhere to the skin or wound, or cause damage to the wound when in contact with the wound. In some embodiments, the dressing 100 may include one or more layers, for example a wound contact layer, beneath the one or more perfluorocarbon layers 102. In some embodiments, the wound dressing 100 may include two or more perfluorocarbon layers. For example, the wound dressing 100 may include 2, 3, 4, 5, 6, 7 or more perfluorocarbon layers.

### Construction of Perfluorocarbon Infused Hydrogel Dressings

In some embodiments, at least some of the layers of the wound dressings 100, may be attached to one another, preventing delamination of the layers. In some embodiments, one or more layers of the wound dressings 100 may include adhesive coating for attachment. In some embodiments, one or more layers of the wound dressings 100 may be tacky or have adhesiveness even without additional adhesive coating, such that they can be attached to the adjacent layers. For example, the layers have adhesive properties, and can be attached to other layers. However, when the layers absorb moisture or wound exudate, the adhesive properties of the layers can be lost or weakened, which can result in delamination of the layers. Accordingly, additional means to secure the layers of the wound dressing are desired. In some embodiments, delamination of layers of a wound dressing may be prevented by physically joining the layers, such as by adhesives, welding, or stitching.

### Experimental Methods

### Sensor Spot Experiments (FIGS. 8A, 8B)

The samples in this experiment include: DURAFIBER incorporating 1.5 g of A2 W18006/P5/916302 Pentafluorophenyltriethoxysilane, DURAFIBER incorporating 3.5 g of B4 W18006/P5/916306 Nonafluorohexyltriethoxysilane, DURAFIBER incorporating 5 g of C4 W18006/P5/916310 1H, 1H,2H,2H-Perfluorodecyltriethoxysilane, and DURAFIBER incorporating 8 g of D5 W18006/P4/910517 1H,1H,2H,2H-Perfluorooxtyltriethoxysilane.

A PSt3 Oxygen Sensor Spot (PreSens, Germany) was fixed to the internal surface of the glass chamber using silicone adhesive, and left to cure in the dark for more than 12 hours, as per the manufacturer's instructions.

The glass chamber was filled using dH₂O ensuring minimal headspace remained. Pure oxygen gas (0.25 ml/min) was bubbled through the liquid via an air stone to ensure gaseous distribution throughout for 2 minutes. A piece of the required dressing (approximately 50 mm×50 mm) was added to the dH₂O and gently pressed to allow submersion to occur and the metal lid screwed as tightly as possible to seal the chamber. The oxygen (% a.s.) was measured and recorded using the contactless Sensor Spots and the associated Fibox 4 meter (PreSens, Germany) The chamber was then stored at room temperature in the dark to prevent Sensor Spot bleaching. The oxygen concentration of the chamber was measured every 60 minutes using the Sensor Spots, and immediately returned to dark conditions. The glass chamber was not opened at any time during the measurement period.

### Sensor Spot Experiments in Parallel (FIGS. 8C-8E)

The samples in this experiment include: DURAFIBER incorporating 1.5 g of A2 W18006/P5/916302 Pentafluorophenyltriethoxysilane, DURAFIBER incorporating 3.5 g of B4 W18006/P5/916306 Nonafluorohexyltriethoxysilane, DURAFIBER incorporating 5 g of C4 W18006/P5/916310 1H,1H,2H,2H-Perfluorodecyltriethoxysilane, and DURAFIBER incorporating 8 g of D5 W18006/P4/910517 1H,1H,2H,2H-Perfluorooxtyltriethoxysilane.

For immersion experiments only, B3 W18006/P5/916306 Nonafluorohexyltriethoxysilane 3.5 g was replaced with B2 W18006/P5/916305 Nonafluorohexyltriethoxysilane 3.5 g due to exhaustion of dressing stock, and D5 W18006/P4/910517 1H,1H,2H,2H-Perfluorooxtyltriethoxysilane 8 g was replaced with D6 W18006/P4/910518 1H,1H,2H,2H-Perfluorooxtyltriethoxysilane 4 g due to exhaustion of dressing stock.

A PSt3 Oxygen Sensor Spot (PreSens, Germany) was fixed to the internal surface of each glass chamber using silicone adhesive, and left to cure in the dark for more than 12 hours, as per the manufacturer's instructions. Each glass chamber (approximately 200 ml volume) was filled using dH₂O ensuring minimal headspace remained. Pure oxygen gas (0.25 ml/min) was bubbled through the liquid via an air stone to ensure gaseous distribution throughout for 2 minutes. A piece of the required dressing (approximately 50 mm×50 mm) was added to the dH₂O and gently pressed to allow submersion to occur and the metal lid screwed as tightly as possible to seal the chamber. The oxygen (% a.s.) of each chamber was measured and recorded sequentially using the contactless Sensor Spots and the associated Fibox 4 meter (PreSens, Germany). The chambers were then stored at room temperature in the dark to prevent Sensor Spot bleaching. The oxygen concentration of each chamber was measured every 60 minutes using the Sensor Spots, and immediately returned to dark conditions. The glass chambers were not opened at any time during the measurement period.

For experiments which featured water immersion, following oxygenation and dressing introduction the vessels were sealed, wrapped in a layer of parafilm and added to the container containing distilled water. The water level was maintained such that the surface of the vessel lids were completely below the surface of the surrounding liquid. These filled containers (sealed with their own lids) were stored in dark conditions until measurements were required.

Before all measurements, the external surface of the dressing vessels was carefully wiped to remove excess liquid. Measurements were then taken as described above, and the vessels were returned to their submersion in the dark.

### Terminology

Any patents and applications and other references noted above, including any that may be listed in accompanying filing papers, are incorporated herein by reference. Aspects of the disclosure can be modified, if necessary, to employ the systems, functions, and concepts of the various references described herein to provide yet further implementations.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Any of the embodiments described herein can be used with a canister or without a canister. Any of the dressing embodiments described herein can absorb and store wound exudate.

The scope of the present disclosure is not intended to be limited by the description of certain embodiments and may be defined by the claims. The language of the claims is to be interpreted broadly based on the language employed in the claims and not limited to the examples described in the present specification or during the prosecution of the application, which examples are to be construed as non-exclusive.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

Certain embodiments of the disclosure are encompassed in the claims presented at the end of this specification, or in other claims presented at a later date.

1. A wound dressing assembly for treating a wound, comprising:
a perfluorocarbon layer configured to deliver oxygen to a wound, the perfluorocarbon layer comprising perfluorocarbon bound to a cellulose fiber.

2. The wound dressing assembly of claim 1, further comprising a cover layer configured to form a seal around the wound.

3. The wound dressing assembly of claim 1, further comprising a membrane electrolytic assembly configured to deliver oxygen to the perfluorocarbon layer.

4. The wound dressing assembly of claim 3, further comprising a wound contact layer.

5. The wound dressing assembly of claim 4, further comprising an absorbent layer.

6. The wound dressing assembly of claim 5, further comprising a transmission layer.

7. The wound dressing assembly of claim 6, further comprising an obscuring layer.

8. The wound dressing assembly of claim 1, wherein the perfluorocarbon layer comprises nonafluorohexyltriethoxysilane.

9. The wound dressing assembly of claim 1, wherein the perfluorocarbon layer contains pentafluorophenyltriethoxysilane.

10. The wound dressing assembly of claim 1, wherein the perfluorocarbon layer contains 1H, 1H, 2H, 2H-perfluorodecyltriethoxysilane.

11. The wound dressing assembly of claim 1, wherein the one or perfluorocarbon layer contains 1H, 1H, 2H, 2H-perfluorooctyltriethoxysilane.

12. The wound dressing assembly of claim 1, wherein the perfluorocarbon layer comprises a gel.

13. The wound dressing assembly of claim 1, wherein the perfluorocarbon layer comprises a hydrogel.

14. The wound dressing assembly of claim 3, wherein the perfluorocarbon layer is attached to the membrane electrolytic assembly.

15. A method for treating a wound comprising:
applying a wound dressing to a wound, the wound dressing comprising a perfluorocarbon layer;
transporting oxygen to the perfluorocarbon layer such that the perfluorocarbon layer stores the oxygen; and
delivering oxygen to the wound from the perfluorocarbon layer.

16. The method of claim 15, wherein the oxygen is provided by a membrane electrolytic assembly.

17. A kit for treating a wound comprising:
a perfluorocarbon layer configured to delivery oxygen to a wound;
a wound dressing configured to distribute oxygen; and
a membrane electrolytic assembly.

18. The kit of claim 17, further comprising a sealing strip.
